SEQUENCE LISTING (41 sequences)

SEQ ID NO: 1 - protein, 10 aa, Artificial Sequence
  Cargo Peptide
KKRKKQKKRK

SEQ ID NO: 2 - protein, 9 aa, Artificial Sequence
  Cargo Peptide
RRKKKQKKK

SEQ ID NO: 3 - protein, 8 aa, Artificial Sequence
  Cargo Peptide
KKQKKRRK

SEQ ID NO: 4 - protein, 9 aa, Artificial Sequence
  Cargo Peptide
KKQKKRRKK

SEQ ID NO: 5 - protein, 8 aa, Artificial Sequence
  Cargo Peptide
KKKQKRKK

SEQ ID NO: 6 - protein, 9 aa, Artificial Sequence
  Cargo Peptide
RQKKQKKKR

SEQ ID NO: 7 - protein, 10 aa, Artificial Sequence
  Cargo Peptide
RKQKKKRKKK

SEQ ID NO: 8 - protein, 9 aa, Artificial Sequence
  Cargo Peptide
KRKQKQKKK

SEQ ID NO: 9 - protein, 9 aa, Artificial Sequence
  Cargo Peptide
KKRKQKKQK

SEQ ID NO: 10 - protein, 8 aa, Artificial Sequence
  Cargo Peptide
KKKRKKQK

SEQ ID NO: 11 - protein, 9 aa, Artificial Sequence
  Cargo Peptide
RKKKKQKKK

SEQ ID NO: 12 - protein, 8 aa, Artificial Sequence
  Cargo Peptide
KKRKKQKK

SEQ ID NO: 13 - protein, 10 aa, Artificial Sequence
  Cargo Peptide
QKKRRKKKQK

SEQ ID NO: 14 - protein, 9 aa, Artificial Sequence
  Cargo Peptide
KKRKQKKRK

SEQ ID NO: 15 - protein, 10 aa, Artificial Sequence
  Cargo Peptide
KKRKQKKQKR

SEQ ID NO: 16 - protein, 10 aa, Artificial Sequence
  Cargo Peptide
KRKQKQKKKK

SEQ ID NO: 17 - protein, 9 aa, Artificial Sequence
  Cargo Peptide
KKRKRKQKK

SEQ ID NO: 18 - protein, 9 aa, Artificial Sequence
  Cargo Peptide
KQKRKKKQK

SEQ ID NO: 19 - protein, 10 aa, Artificial Sequence
  Cargo Peptide
KQKKRQKKKR

SEQ ID NO: 20 - protein, 10 aa, Artificial Sequence
  Cargo Peptide
KKKRKQKQKK

SEQ ID NO: 21 - protein, 10 aa, Artificial Sequence
  Cargo Peptide
RKKKQKKQKK

SEQ ID NO: 22 - protein, 9 aa, Artificial Sequence
  Cargo Peptide
KKKRQKKQK

SEQ ID NO: 23 - protein, 10 aa, Artificial Sequence
  Cargo Peptide
KKRKKKKKRK

SEQ ID NO: 24 - protein, 8 aa, Artificial Sequence
  Cargo Peptide
RRKKKKKK

SEQ ID NO: 25 - protein, 7 aa, Artificial Sequence
  Cargo Peptide
KKKKRRK

SEQ ID NO: 26 - protein, 8 aa, Artificial Sequence
  Cargo Peptide
KKKKRRKK

SEQ ID NO: 27 - protein, 7 aa, Artificial Sequence
  Cargo Peptide
KKKKRKK

SEQ ID NO: 28 - protein, 8 aa, Artificial Sequence
  Cargo Peptide
KKRKKKKK

SEQ ID NO: 29 - protein, 10 aa, Artificial Sequence
  Cargo Peptide
KKRKKHKKRK

SEQ ID NO: 30 - protein, 9 aa, Artificial Sequence
  Cargo Peptide
RRKKKHKKK

SEQ ID NO: 31 - protein, 8 aa, Artificial Sequence
  Cargo Peptide
KKHKKRRK

SEQ ID NO: 32 - protein, 9 aa, Artificial Sequence
  Cargo Peptide
KKHKKRRKK

SEQ ID NO: 33 - protein, 8 aa, Artificial Sequence
  Cargo Peptide
KKKHKRKK

SEQ ID NO: 34 - protein, 9 aa, Artificial Sequence
  Cargo Peptide
RHKKHKKKR

SEQ ID NO: 35 - protein, 10 aa, Artificial Sequence
  Cargo Peptide
RKHKKKRKKK

SEQ ID NO: 36 - protein, 9 aa, Artificial Sequence
  Cargo Peptide
HHKRKKKRK

SEQ ID NO: 37 - protein, 8 aa, Artificial Sequence
  Cargo Peptide
KKRHHKRK

SEQ ID NO: 38 - protein, 7 aa, Artificial Sequence
  Cargo Peptide
KKHRKKH

SEQ ID NO: 39 - protein, 7 aa, Artificial Sequence
  Cargo Peptide
KKKQKRK

SEQ ID NO: 40 - protein, 8 aa, Artificial Sequence
  Control  peptide
KKKKKKKK

SEQ ID NO: 41 - protein, 12 aa, Artificial Sequence
  Control peptide
ARPLEHGSDKAT

(19) United States
(12) Patent Application Publication
(10) Pub. No.: US 2007/0086981 A1
(43) Pub. Date: Apr. 19, 2007

(54) Delivery peptides, their constructs with active agents and use

(76) Inventors: Hans Meijer, DUBAI (AE); Peter Wehling, Dusseldorf (DE)

Correspondence Address: OSTROLENK FABER GERB & SOFFEN, 1180 AVENUE OF THE AMERICAS, NEW YORK, NY, 100368403, US

(21) Appl. No.: 10/579,941

(22) PCT Filed: Nov. 19, 2004
(86) PCT No.: PCT/EP04/13203

§ 371 Date: Oct. 16, 2006

(30) Foreign Application Priority Data
Nov. 21, 2003 (DE) 103 55 559.5

(51) Int. Cl. A61K 48/00; A61K 38/14; A61K 38/08; C07K 7/06

(52) U.S. Cl. 424/93.2; 514/8; 514/16; 530/328; 530/329; 977/802

(57) ABSTRACT

The present invention refers to delivery proteins, protein-cargo complexes, methods, and means for the enhanced delivery or transport of drugs, biologically active agents or other compounds as cargo or cargo molecules onto, into, or across biological membranes or tissues forming a biological barrier.

The present invention refers to compositions, methods, and means for the enhanced delivery or transport of drugs, biologically active agents or other compounds as cargo or cargo molecules onto, into, or across biological membranes or tissues forming a biological barrier. Biological barriers include for example, cell membranes, mitochondrial membranes, and dermal and epithelial membranes like the skin, the gastrointestinal epithelium, and the bronchial or pulmonary epithelium, as well as endothelial membranes like the blood brain barrier.

The capability to deliver biologically active substances such as drugs, therapeutic agents, nucleic acids, amino acids, small molecules, viruses etc. across and/or onto the surface of or into biological barriers or specific cell types is useful for various applications, in particular for the therapy of the animal or human body, particularly in the fields of oncology, gene therapy and in connection with the prophylaxis and/or therapy of gastrointestinal diseases or skin diseases.

Most small-moleculed drugs for example reach their targets as they are able to passively diffuse through the cell membrane. But reliance on passive diffusion limits the universe of drugs to those that are soluble in both the polar extracellular environment and the non-polar cell membrane. Therefore, there is also a need for the provision of means and methods to deliver all types of biologically active substances through the cell membrane, without relying on passive diffusion.

Protein transduction is a recently discovered process, wherein small proteins, in particular polypeptides carrying short specific sequences, also named Protein Transduction Domains (PTDs), are used to deliver not only proteins, but also great varieties of different molecules which are covalently linked to such a “delivery” peptide. Several naturally occurring proteins have been found to enter cells easily and can therefore serve as delivery proteins, including the TAT protein from the human immunodeficiency virus (HIV), the antennapedia protein from drosophila, and the VP22 protein from the herpes simplex virus. Specific short sequences with amphiphilic structure within the larger molecule account for most of the transduction abilities of these proteins.

There are also approaches to design artificial proteins to function as delivery proteins. For example, peptide sequences have been shown to get into the cell plasma membrane as well as across the stratum corneum of the skin (Nature Medicine (2000) 6:1253). Although the plasma membrane of a cell is different from the biological barrier constituted by the stratum corneum of the skin, both barriers can be crossed by such delivery peptides.

The WO 02/069930 A1 relates to compositions and methods for enhancing drug delivery across and into tissues, including the skin, gastrointestinal tract, pulmonary epithelium, and the blood brain barrier, and describes the provision of a delivery-enhancing transporter protein comprising fewer than 50 sub-units and at least 5 guanidino- or amidino-moieties, e.g. arginine (R), which are covalently linked to a biologically active compound to be transported across the barrier.

The WO 01/15511 A2 relates to so called “internalising” peptides, which facilitate the uptake and transport of cargo into the cytoplasm and the nuclei of cells. These peptides were obtained by M13-phage library screening with HIG-82 cells, human primary T cells, cells of a human epithelial cell line or human cervical mucosa tissue. A great variety of different peptides (75 peptides) were found, which may serve as delivery enhancing internalising peptides.

The WO 01/62297 A1 relates to compositions and methods for enhancing the delivery of drugs and other agents across biological barriers, such as the skin, gastrointestinal tract, pulmonary epithelium and the blood brain barrier by providing delivery-enhancing polymers including, for example poly-arginine molecules between about 6 and 50 residues in length.

For example, as the skin is a natural barrier for substances to protect the whole body, transdermal transport of substances is difficult. Despite many years of research only few drugs are known, which able to penetrate into the deeper layers of skin. Most of these substances, like DMSO, also function as solvents and include the risk of health damage. Thus, an efficient transdermal transport mechanism is highly desirable for the application of therapeutic and cosmetic agents for skin care, skin protection and the treatment of skin disease. A good example of skin care is the local anti-aging treatment of skin. At present no creams, in particular skin creams, are available which can effectively prevent the formation of wrinkles in the face and create a young condition of the skin. It is well known that skin aging does not only concern the dermis but also the deeper layers. Effective anti-aging products must have a broad activity spectrum in all skin layers. For slowing or even stopping the changes in the composition of the matrix proteins, which are responsible for the skin firmness and elasticity, cosmetic agents have also to be transported into the most deep layers of the skin. For current skin creams it has not been shown, that the anti-aging agents are transported even into the deeper layers. Thus a transport mechanism, which can bring active anti-aging agents into the deeper layers, is highly desirable.

Many substances, for example vitamins, retinoic acid, hyaluronic acid and collagen, can inhibit the changes in the composition of the matrix proteins and thus have anti-aging activity. The application of proteins is highly desirable as proteins have a high specific biological activity. A good example for a protein with anti-aging activity is Super-Oxid- Dismutase (SOD), a free radical catcher. Free radicals increase inflammation and lead to large number of different processes and diseases in all kinds of tissues, like the aging process in the skin. In human skin the inflammation affects negatively the composition of the matrix proteins, leads to a loss of firmness and elasticity and finally to the formation of wrinkles. SOD has been shown to catch free radicals specifically and to inhibit cell death and thus will have anti-aging activity, if it is transported into all layers of the skin.

For effective skin care, skin protection and the treatment of skin disease and epidermal diseases in general, an effective method is needed for the transportation of active substances, specifically of biologically active proteins like SOD, into the deeper layers of the skin. These substances strongly differ in their physical and chemical properties. Ideally, one general method can be applied to all substances.

As mentioned, the transdermal transport can be enhanced by the application of delivery peptides conjugated to a cargo molecule. Yet these particular peptides are not able to enhance the transdermal transport of different kinds of cargo in general and an inhibition of the biological activity of the cargo to be transported cannot be ruled out. It would be advantageous, to have PTDs which can transport various kinds of cargo molecules and which do not have to be removed for the restoration of the biological activity of the transported cargo molecule.

In known delivery proteins a significant portion of the topological surface of a cargo molecule associated with the protein is often involved. It is therefore necessary, for biological activity, the cargo portion of the protein-cargo complex be severed from the attached delivery protein after crossing the biological barrier or entering the target cell and free drug be released after passing through a biological barrier.

Accordingly, the technical problem underlying the present invention basically is to provide methods and means for an enhanced delivery of a great variety of employable biologically active substances as cargo onto the surface of, into or across biological barriers, without considerable reduction or inhibition of the biological activity of the cargo conjugated to the delivery peptide. Most preferably, the biological activity of the cargo is basically unaltered compared to the biological activity of the cargo in the absence of the delivery peptide.

The technical problem underlying the present invention is solved by the provision of a polypeptide functioning as a delivery peptide and belonging to a family of peptides sharing the general formula I:
(K)ₙA₁B₁C₁(K)ₘA₂B₂C₂(K)ₗA₃B₃C₃(K)ₒ, (I)
wherein
K is lysine (K),
n, m, l, o is an integer from 0 to 5,
B₁, B₂, B₃ is arginine (R), glutamine (Q) or histidine (H),
A₁, A₂, A₃, C₁, C₂, C₃ is arginine (R), histidine (H) or is missing, and
the total number of amino acid residues is not more than 10.

One embodiment of the invention is a peptide comprising at least one amino acid sequence according to formula I. In a preferred embodiment the peptide consists of the amino acid sequence of formula I.

In a further preferred embodiment the peptide comprises, in particular consists of, an amino acid sequence selected from the group consisting of:

| | |
|---|---|
| KKRKKQKKRK, | (SEQ ID NO:1) |
| RRKKKQKKK, | (SEQ ID NO:2) |
| KKQKKRRK, | (SEQ ID NO:3) |
| KKQKKRRKK, | (SEQ ID NO:4) |
| KKKQKRKK, | (SEQ ID NO:5) |
| RQKKQKKKR, | (SEQ ID NO:6) |
| RKQKKKRKKK, | (SEQ ID NO:7) |
| KRKQKQKKK, | (SEQ ID NO:8) |
| KKRKQKKQK, | (SEQ ID NO:9) |
| KKKRKKQK, | (SEQ ID NO:10) |
| RKKKKQKKK, | (SEQ ID NO:11) |
| KKRKKQKK, | (SEQ ID NO:12) |
| QKKRRKKKQK, | (SEQ ID NO:13) |
| KKRKQKKRK, | (SEQ ID NO:14) |
| KKRKQKKQKR, | (SEQ ID NO:15) |
| KRKQKQKKKK, | (SEQ ID NO:16) |
| KKRKRKQKK, | (SEQ ID NO:17) |
| KQKRKKKQK, | (SEQ ID NO:18) |
| KQKKRQKKKR, | (SEQ ID NO:19) |
| KKKRKQKQKK, | (SEQ ID NO:20) |
| RKKKQKKQKK, | (SEQ ID NO:21) |
| KKKRQKKQK, | (SEQ ID NO:22) |
| KKRKKKKKRK, | (SEQ ID NO:23) |
| RRKKKKKK, | (SEQ ID NO:24) |
| KKKKRRK, | (SEQ ID NO:25) |
| KKKKRRKK, | (SEQ ID NO:26) |
| KKKKRKK, | (SEQ ID NO:27) |
| KKRKKKKK, | (SEQ ID NO:28) |
| KKRKKHKKRK, | (SEQ ID NO:29) |
| RRKKKHKKK, | (SEQ ID NO:30) |
| KKHKKRRK, | (SEQ ID NO:31) |
| KKHKKRRKK, | (SEQ ID NO:32) |
| KKKHKRKK, | (SEQ ID NO:33) |
| RHKKHKKKR, | (SEQ ID NO:34) |
| RKHKKKRKKK, | (SEQ ID NO:35) |
| HHKRKKKRK, | (SEQ ID NO:36) |
| KKRHHKRK, | (SEQ ID NO:37) |
| KKHRKKH | (SEQ ID NO:38) |
| and | |
| KKKQKRK. | (SEQ ID NO:39) |

In a further preferred embodiment the peptide solely consists of amino acids selected from the group of amino acids consisting of histidine (H), lysine (K), glutamine (Q), and arginine (R).

The peptide according to the present invention, in particular delivery peptides, which transport, deliver, or facilitate or enhance transport or delivery and, most advantageously, do not affect or hinder the biological activity of the cargo molecule. Most advantageously, the biological activity of the molecule associated with the delivery peptide, forming a peptide-cargo conjugate or complex, is substantially the same as of the isolated cargo in the absence of the delivery peptide. Most advantageously, there is no need, for the cargo to become active, to cleave off the delivery peptide as soon as the peptide-cargo conjugate or complex has been internalised into the target tissue or cell or has reached or crossed the biological barrier towards its target.

The present invention provides the above peptides preferably in isolated and/or purified form.

In contrast to known delivery peptides, for example from WO 91/09958 or WO 02/069930, the embodied peptide does not affect the biological activity of cargo molecules.

Furthermore, the delivery of the cargo in the form of a peptide-cargo conjugate or complex is enhanced or increased compared to the delivery of the cargo in the absence of the delivery peptide. For example, the peptide according to the invention enhances the transport of proteins, e.g. therapeutically active enzymes, into the deeper layers of the skin.

Without being committed to the theory, the peptide according to the invention exhibits enhanced translocation activity due to the presence of at least one amino acid with basic charge, exhibits enhanced translocation activity, mainly due to the fact that amino acids in the delivery peptide form a regular stable sheet, and confers to a cargo molecule no reduction in its biological activity due to the fact that the peptide does not or only to a little extend interact with the cargo molecule. Interaction between the cargo molecule and the sheet formed by the peptide according to the invention like steric hindrance and too strong basic charge is mainly reduced.

Without being committed to the theory, glutamine (Q) present within a preferred embodiment of the peptide stabilises the sheet because of the polar interaction; lysine (K) contributes more to a stable sheet than arginine (R); histidine (H) present within a preferred embodiment of the peptide contributes to the basic charge upon presence of low pH.

Delivery peptides can be constructed by any method known in the art. Preferably, delivery peptide polymers are produced synthetically, preferably using a commercially available peptide synthesizer. N-methyl and hydroxy-amino acids can be substituted for conventional amino acids in solid phase peptide synthesis. In a preferred embodiment biotinylated peptides are produced. In another preferred embodiment free amino terminal groups are capped with a blocking group, such as an acetyl or benzyl group, in particular to prevent ubiquitination in vivo.

The invention also embodies a complex that includes at least one peptide according to the invention and at least one cargo molecule, in the following referred to as peptide-cargo complex. The embodied delivery peptides are well suited for the surface coverage of cargo molecules. In presence of at least one of the peptide according to the invention the uptake of the at least one cargo molecule is strongly enhanced in comparison to the uptake of a cargo molecule without the peptide. The delivery technology has broad potential for enhancing the penetration of a wide range of therapeutic agents into and a cross skin and other biological barriers.

Most advantageously, common amounts of solubilising agents such as CREMOPHOR® EL (from BASF; mainly polyoxyethylated castor oil), polysorbate 80 (polyoxyethylene sorbitan monooleate, also known as TWEEN 80), PEG, and ethanol are not required with the embodied delivery peptides. Accordingly, side-effects typically associated with these solubilising agents, such as anaphylaxis, dyspnea, hypotension, and the like, can be greatly reduced.

Accordingly, the technical problem is further solved by the provision of a peptide-cargo complex comprising the delivery peptide and at least one cargo molecule. In a preferred embodiment of the peptide-cargo complex the delivery peptide is selectively linked to the outer surface of at least one cargo molecule.

The delivery peptides of the present invention may be complexed with cargo or at least one cargo molecule. The term “cargo” or “cargo molecule”, as used herein, refers to any small molecule, macromolecule or macromolecular complex which may be useful to transfer onto, across or into a biological barrier or a target cell.

Preferably, cargo includes, but is not limited to, small organic molecules, marcomolecules, polynucleotides, DNA, oligonucleotide decoys, RNA, antisense RNA and other antisense constructs, polypeptides, proteins, viruses, modified viruses, viral and non-viral vectors, metals, and plasmids. Accordingly, in a preferred embodiment of the invention the cargo comprises at least one compound selected from the group consisting of polynucleotides, polypeptides, proteins, small organic molecules, metals, viruses, modified viruses, viral vectors, and plasmids. Preferably, the cargo is a virus selected from the group consisting of adenoviruses, adeno-associated viruses, herpes viruses, simplex virus, and retroviruses.

The delivery peptides of the present invention are preferably linked to cargo by any known method, such as, but not limited to, chemical cross-linking, avidin bridge, glutation-S-transferase bridge, a linkage comprising at least one disulfide bond, peptide-cargo fusion proteins etc.

Preferably, various functional groups, such as hydroxyl, amino, halogen, etc. present on the cargo are used as a handle to attach a suitable complexing group. For example, a hydroxyl group is modified to include an acidic phosphate group.

In another preferred variant the linkage includes a disulfide bond. In another variant the linkage includes a streptavidin-biotin complex. Preferably, the delivery peptide is biotinylated and the cargo molecule is avidin labelled.

In a preferred embodiment the delivery peptides of the present invention are linked to cargo by a cleavable linker.

In some preferred embodiments, the cargo is modified to incorporate a functional group, such as a carboxylic acid group, a phosphate or phosphate ester, a sulfonic acid group, and the like. Preferably, the cargo is modified to incorporate a suitable group by attaching the group via a linker to the cargo. Preferably, the linker will be a cleavable linker which can liberate the biological agent. Most preferably, the cargo is modified using a number of methods known in the art, either directly, e.g. with a carbodiimide, or via at least one linking moiety. In particular, carbamate, ester, thioether, disulfide, and hydrazone linkages are formed. Ester and disulfide linkages are preferred, if the linkage is to be readily degraded in the cytosol, after transport of the substance across the cell membrane.

Although the present invention relates to advanced delivery proteins which confer a minimum or even no interaction with the cargo, in some rare cases a significant portion of the topological surface of a cargo molecule is often involved in the protein-cargo conjugation. It is therefore required, for biological activity, the cargo portion of the protein-cargo complex may need to be severed from the attached delivery protein and linker moiety (if any) for the cargo to exert biological activity after crossing the biological barrier or entering the target cell. For such situations, the protein-cargo complex or conjugate preferably includes a cleavable linker for releasing free drug after passing through the biological barrier or the cell membrane.

In a preferred embodiment of the present invention, cargo is essentially any biologically active agent or diagnostic molecule. In some preferred embodiments, the biologically active agent is used in its unmodified form, while in other preferred embodiments, the agent will be modified to incorporate a charged (typically acidic) residue to enhance the peptide-cargo complex. The term “biologically active agent” as used herein includes agents in their unmodified form as well as agents that have been modified, for example prodrugs, and have reduced or augmented levels of activity and/or reduced or augmented binding kinetics compared with the parent agent.

Small organic molecules, also named small molecules, are therapeutically useful and preferably include drugs or other biologically or therapeutically active agents which act to ensure proper functioning of a cell or molecules which may induce apoptosis or cell lysis, where death of a cell, such as a cancerous cell, is desired. The subject of the invention is most advantageous for delivering small organic molecules that have poor solubilities in aqueous liquids, such as serum and aqueous saline. Thus, compounds whose therapeutic efficacies are limited by their low solubilities can be administered in greater dosages according to the present invention, and can be more efficacious on a molar basis in combined form, relative to the non-combined form, due to higher uptake levels by cells.

Exemplary of such small organic molecules that form preferred compositions according to the present methods are the taxanes. The complex has enhanced trans-epithelial tissue transport rates relative to corresponding non-complexed forms and is particularly useful for inhibiting growth of cancer cells. Taxanes and taxoids are believed to manifest their anticancer effects by promoting polymerisation of microtubules (and inhibiting depolymerisation) to an extent that is deleterious to cell function, inhibiting cell replication and ultimately leading to cell death. As used herein, the term “taxane” refers to paclitaxel, F, R′=acetyl, R″=benzyl, also known under the trademark “TAXOL”, and naturally occurring, synthetic, or bioengineered analogs. Preferably, the delivery peptide is combined with a modified taxane or taxoid which has been modified to include an acid moiety, e.g. phosphate.

Given as a further example, highly charged agents, such as levodopa (L-3,4-dihydroxy-phenylalanine; L-DOPA) are combined as cargo with the delivery protein of the invention. Peptoid and peptidomimetic agents are also contemplated as cargo.

In a preferred embodiment macromolecules as cargo are transported across one or more layers of an epithelial or endothelial tissue, which is exemplified by proteins, and in particular, enzymes. Therapeutic proteins as cargo include, but are not limited to replacement enzymes.

Given as an example, therapeutic enzymes as cargo include, but are not limited to, alglucerase, for use in treating lysosomal glucocerebrosidase deficiency (Gaucher's disease), alpha-L-iduronidase, for use in treating mucopolysaccharidosis I, alpha-N-acetylglucosamidase, for use in treating sanfilippo B syndrome, lipase, for use in treating pancreatic insufficiency, adenosine deaminase, for use in treating severe combined immunodeficiency syndrome, and triose phosphate isomerase, for use in treating neuromuscular dysfunction associated with triose phosphate isomerase deficiency.

In another preferred embodiment the delivery peptide is combined with a wide variety of cargo that has diagnostic use. Preferably, cargo is a diagnostic imaging or contrast agent, which is most advantageuosly delivered according to the invention onto, into or across one or more layers of an biological barrier. In a preferred embodiment the delivery peptide is labelled with radioactivity, such as “mTc glucoheptonate, or substances used in magnetic resonance imaging (MRI) procedures such as gadolinium doped chelation agents, e.g. Gd-DTPA. Other examples of diagnostic agents employed as cargo include marker genes that encode proteins that are readily detectable when expressed in a cell including, but not limited to, β-galactosidase, green fluorescent protein, luciferase, and the like. In particular, cargo is selected from metals, halogens radionuclides, fluors, enzymes, enzyme substrates, enzyme cofactors, enzyme inhibitors, ligands (particularly haptens), and the like.

The present invention also provides compositions and methods for various classes of therapeutic agents. In a preferred embodiment cargo is a agent that can be combined with the delivery protein according to the invention to greatly improve the agent's tissue penetration and efficacy are compounds such as antibacterial agents, antifungal agents, antiviral agents, antiproliferative agents, immunosuppressive agents, vitamins, analgesics, hormones and the like.

Accordingly, a preferred embodiment the cargo is selected from the group consisting of therapeutic proteins, suicide proteins, tumour suppressor proteins, transcription factors, kinase inhibitors, kinases, regulatory proteins, apoptotic proteins, anti-apoptotic proteins, viral antigens, cellular antigens, differentiation factors, immortalisation factors, toxines, enzymes, nucleic acids, antisense constructs, diagnostic imaging or contrast agents, dyes, antibacterial agents, antifungal agents, antiviral agents, antiproliferative agents, cytostatics, immunosuppressive agents, vitamins, analgesic agents, hormones, anti-inflammatory agents, and anti-aging agents.

In a preferred embodiment cargo is an antibacterial agent which is used in the present compositions and methods. Such cargo include the β-lactam antibiotics and quinolone antibiotics. Preferably, the cargo is an antibacterial agent selected from the group consisting of nafcillin, oxacillin, penicillin, amoxacillin, ampicillin, cephalosporine, cefotaxime, ceftriaxone, rifampin, minocycline, ciprofloxacin, norfloxacin, erythromycin, tetracycline, gentamicin, a macrolide, a quinolone, a β-lactone, a P-lactamase inhibitor, salicylamide, and vancomycin. More particularly, the cargo is selected from nafcillin, oxacillin, penicillin, amoxacillin, ampicillin, cefotaxime, ceftriaxone, rifampin, minocycline, ciprofloxacin, norfloxacin, erythromycin, vancomycin, and analogs thereof.

In another preferred embodiment cargo is an antimicrobial agent that is used in the present compositions and methods. Preferably, cargo is selected from sulfanilamide, sulfamethoxazole, sulfacetamide, sulfisoxazole, sulfadiazine, penicillins such as penicillins G and V, methicillin, oxacillin, naficillin, ampicillin, amoxacillin, carbenicillin, ticarcillin, mezlocillin and piperacillin, cephalosporins such as cephalothin, cefaxolin, cephalexin, cefadroxil, cefamandole, cefoxitin, cefaclor, cefuroxine, loracarbef, cefonicid, cefotetan, ceforanide, cefotaxime, cefpodoxime, proxetil, ceftizoxime, cefoperazone, ceftazidime and cefepime, aminoglycosides such as gentamycin, tobramycin, amikacin, netilmicin, neomycin, kanamycin, streptomycin, and the like, tetracyclines such as chlortetracycline, oxytetracycline, demeclocycline, methacycline, doxycycline and minocycline, and macrolides such as erythromycin, clarithromycin, and azithromycin.

In another preferred embodiment cargo is an antifungal agent that is used in the present compositions and methods. Preferably, cargo is selected from amphotericin, itraconazole, ketoconazole, miconazole, nystatin, clotrimazole, fluconazole, ciclopirox, econazole, naftifine, terbinafine, and griseofulvin.

In another preferred embodiment cargo is an antiviral agent that is used in the present compositions and methods. Preferably, cargo is selected from aciclovir, famciclovir, ganciclovir, foscarnet, idoxuridine, sorivudine, trifluridine (trifluoropyridine), valacyclovir, cidofovir, didanosine, stavudine, zalcitabine, zidovudine, ribavirin, and rimantatine.

In another preferred embodiment cargo is an antiproliferative or immunosuppressive agent that is used in the present compositions and methods. Preferably, cargo is selected from methotrexate, azathioprine, fluorouracil, hydroxyurea, 6-thioguanine, cyclophosphamide, mechloroethamine hydrochloride, carmustine, cyclosporine, taxol, tacrolimus, vinblastine, dapsone, nedocromil, cromolyn (cromoglycic acid), and sulfasalazine.

In another preferred embodiment cargo is an histamine receptor agonist or antagonist. Preferably, cargo is selected from 2-methylhistamine, 2-pyridylethylamine, 2-thiazolylethylamine, (R)-a-methylhistamine, impromidine, dimaprit, 4(5)-methylhistamine, diphenhydramine, pyrilamine, promethazine, chlorpheniramine, chlorcyclizine, terfenadine, and the like.

In another preferred embodiment cargo is an agent useful in treating asthma. Preferably, cargo is selected from the corticosteroids including beclomethasone, budesonide, and prednisone, as well as cromolyn, nedocromil, albuterol, bitolterol mesylate, pirbuterol, salmeterol, terbutyline and theophylline.

In another preferred embodiment cargo is a vitamin.

In another preferred embodiment cargo is an analgesic agent including, lidocaine, bupivacaine, novocaine, procaine, tetracaine, benzocaine, cocaine, mepivacaine, etidocaine, proparacaine ropivacaine, prilocaine and the like.

In another preferred embodiment cargo is an antineoplastic agent that is used in the present compositions and methods. Preferably, cargo is selected from pentostatin, 6-mercaptopurine, 6-thioguanine, methotrexate, bleomycins, etoposide, teniposide, dactinomycin, daunorubicin, doxorubicin, mitoxantrone, hydroxyurea, 5-fluorouracil, cytarabine, fludarabine, mitomycin, cisplatin, procarbazine, dacarbazine, paclitaxel, colchicine, vinca alkaloids, and the like.

In another preferred embodiment cargo is a hormone selected from the group consisting of tissue hormones, in particular prostaglandine, serotonine, histamine, bradykinin, kallikrein, and gastrointestinal hormones, releasing hormones, pituitary hormones, insulin, vasopressin (ADH), glucagon, enkephalin, calcitonin, and corticosteroides.

In another preferred embodiment the delivery peptide is combined with nucleic acids as cargo, which code for a protein, RNA, ribosome, or antisense RNA. The protein, RNA or ribosome encoded by the nucleid acid may be under-represented, defunct or non-existent in the cell and the antisense RNA encoded by the nucleic acid may allow for the elimination of an undesired function of a molecule.

In a preferred embodiment the peptides of the present invention are synthesised as a fusion with a peptide, in particular a peptide nucleic acid (PNA) which is a DNA-mimic capable of forming double and triple helices with DNA. Such peptide-PNA fusion can form a stable DNA or RNA/PNA duplex, which may enter cells via the peptides of the present invention, thereby delivering the DNA or RNA to a target cell.

Accordingly, cargo also include nucleic acids, including oligonucleotides and polynucleotides formed of DNA and RNA, and analogs thereof, which have selected sequences designed for hybridisation to complementary targets, such as antisense sequences for single- or double-stranded targets, or for expressing nucleic acid transcripts or proteins encoded by the sequences. Analogs include charged and preferably uncharged backbone analogs, such as phosphonates, methyl phosphonates, phosphoramidates, preferably N-3′ or N-5′, thiophosphates, uncharged morpholino-based polymers, and protein nucleic acids (PNAs). Such molecules can be used in a variety of therapeutic regimens, including enzyme replacement therapy, gene therapy, and antisense therapy, for example.

By way of example, protein nucleic acids (PNA) are analogs of DNA in which the backbone is structurally homomorphous with a deoxyribose backbone. The backbone consists of N-(2-aminoethyl)glycine units to which the nucleobases are attached. PNAs containing all four natural nucleobases hybridise to complementary oligonucleotides obeying Watson-Crick base-pairing rules, and is a true DNA-mimic in terms of base pair recognition (Egholm et al. (1993) Nature 365:566-568). The backbone of a PNA is formed by peptide bonds rather than phosphate esters, making it well-suited for anti-sense applications. Since the backbone is uncharged, PNA/DNA or PNA/RNA duplexes that form exhibit greater than normal thermal stability. PNAs have the additional advantage that they are not recognized by nucleases or proteases. In addition, PNAs can be synthesized on an automated peptides synthesizer using standard t-Boc chemistry. The PNA is then readily linked to a transport polymer of the invention.

In another preferred embodiment the delivery peptide is combined with decoy oligonucleotides which preferably contain specific binding sites for transcription factors and may block the function of the transcription factors in vitro and in vivo.

Where the cargo is a polypeptide, the polypeptide preferably is a peptide or protein which, particularly when delivered across the biological barrier or to the target cell, provides a desired function to the cell, or induces a particular phenotypic alteration. In another preferred embodiment the protein or peptide is an antigen capable of eliciting an immune response within the cell.

The delivery peptides of the present invention are also useful for delivery of cargo into cells in vivo and facilitate, in situ or localised, delivery of cargo in vivo. The delivery peptides of the present invention also facilitate the nuclear translocation of a cargo. In particular, the delivery peptides allow for the co-entry of the peptide-linked cargo, and the translocation of the cargo to the nuclei.

The delivery peptides of the present invention most advantageously facilitate uptake and delivery into a wide variety of cell types including cells which are refractory to virus infection, such as primary human airway epithelial cells of the bronchial or pulmonary epithelium, as well as other types of primary and established cell lines such as synovial cells (human or animal), primary human islet cells, myoblast cells, kidney epithelial cells, fibroblast cells, tumor cells, cells of the different germal layers as well as mucosa, such as cervical mucosa.

As the peptides of the present invention facilitate the uptake of cargo into synovial lining cells in vivo, the peptides are in particular useful in the alleviation of arthritis. One approach to alleviating rheumatoid arthritis in a subject is to induce synovial cell death. The peptides of the present invention can induce apoptosis preferably in synovial cells when linked for example to an apoptosis factor, for example B-53, caspase or an anti-microbial peptide.

The peptides according to the invention are also useful for delivering the apoptosis factor, or a DNA encoding an apoptosis factor to tissues such as arthritic joints and inducing apoptosis therein. The peptides of the present invention are also useful for delivering apoptosis factors to tumor cells and inducing apoptosis therein, in particular in tumor cells previously found in various treatments known in the art to be resistant to apoptosis.

Proteinases, particularly serine proteinases and neutral metallo-proteinases, are involved in the degradiation of articular cartilage. Mesenchymal cells of the articular joint and white blood cells which colonise the joint during an inflammatory response, for example in rheumatoid arthritis, synthesise various proteinases, which degrade articular cartilage. Therefore, reduction of white blood cells at the site of inflammation in arthritic joints is an approach to anti-erosive therapy in arthritis. The delivery peptides of the present invention are useful in delivering apoptotic factors to cells in arthritic joints, including white blood cells, for example in lavage fluid of inflamed arthritic joints, and cause a great reduction of white blood cells, for example in the lavage fluid of IL-1 inflamed joints. White blood cell reduction is also useful to reduce swelling, synovial proliferation and cartilage degradation in arthritic joints.

In another aspect of the present invention the delivery peptides are associated with therapeutic proteins as cargo, which are growth factors, cytokines. In a preferred embodiment the cargo is a therapeutic agent used for a condition selected from the group consisting of inflammatory and degenerative joint and spine diseases, arthritis, especially osteoarthritis, low back pain, bone repair, fracture healing, therapy of muscle and ligament injury. In particular, cargo is selected from IL-1Ra, STNF-R (p55), STNF-R (p75), SIL-1R type I, SIL-1R type II, BMP-2, BMP-6, BMP-7, LMP-1, LMP-3, IGF-1, TGF-β1, TGF-β2, TGF-β3, IL4, IL-10, CTLA4, CD30, TIMP-1, IFN-β, Sox-9, and PDGF. In a preferred embodiment the growth factors and cytokines are administered to the target tissue or cell via the peptides of the present invention alone, but also in conjunction with any other conventional means of administration.

The peptides of the present invention are also useful for developing improved immunogens. For example, the peptides of the present invention advantageously facilitate delivery of proteins, polypeptides, DNA, RNA, vectors and viruses to target cells in an animal or human body, which may be useful as immunogens. The peptide-cargo complexes of the present invention are capable of eliciting an immune response when administered to a target cell of an animal or human body. In one preferred embodiment, such immunogens are vaccines, preferably a vaccine for HIV. In a preferred embodiment the cargo portion of the peptide-cargo complex acting as an immunogen is an antigen such as an HIV envelope protein, gag, pol, env, tat, nef, vpr, vpv, and rev.

In another preferred embodiment, the invention is used for delivering immunospecific antibodies or antibody fragments to the cytosol to interfere with deleterious biological processes such as microbial infection. Recent experiments have shown that intracellular antibodies can be effective antiviral agents in plant and mammalian cells. In contrast to the typically used single-chain variable region fragments (scFv), in which the antibody heavy and light chains are synthesized as a single polypeptide, the delivery peptide of the invention is complexed to such scFv fragments. Most advantageously the degree of cellular uptake is increased, allowing the immunospecific fragments to bind and disable important microbial components, such as HIV Rev, HIV reverse transcriptase, and integrase proteins.

Peptides as cargo also include, effector polypeptides, receptor fragments, and the like. Cargo is preferably selected from peptides having phosphorylation sites used by proteins mediating intra-cellular signals. Such proteins as cargo include, but are not limited to, protein kinase C, RAF-1, p21Ras, NF-KB, C-JUN, and cytoplasmic tails of membrane receptors such as IL-4 receptor, CD28, CTLA-4, V7, and MHC Class I and Class II antigens.

Preferably, the process for eliciting an immune response in an animal or human body is accomplished by ex vivo transduction of target cells followed by the presentation of the transduced cells into a subject, for example, intramuscular, or intra-dermal injection.

In one preferred embodiment, the protein antigen is a tumor antigen for eliciting or promoting an immune response against tumor cells. The transport of isolated or soluble proteins into the cytosol of APC with subsequent activation of CTL is exceptional, since, with few exceptions, injection of isolated or soluble proteins does not result either in activation of APC or induction of CTLs. Thus, antigens that are complexed with the transport enhancing compositions of the present invention can serve to stimulate a cellular immune response in vitro or in vivo.

According to a further aspect of the invention, protein antigens as cargo are delivered to the cytosolic compartment of antigen-presenting cells (APCs), where they are degraded into peptides. The peptides are then transported into the endoplasmic reticulum, where they associate with nascent HLA class I molecules and are displayed on the cell surface. Such “activated” APCs can serve as inducers of class I restricted antigen-specific cytotoxic T-lymphocytes (CTLs), which then proceed to recognize and destroy cells displaying the particular antigen. APCs that are able to carry out this process include, but are not limited to, certain macrophages, B cells and dendritic cells.

In a preferred embodiment, the process for eliciting an immune response in an animal or human body comprises administering to a target cell, in vitro, in vivo, or ex vivo, a peptide-cargo complex of the present invention, wherein the cargo is an antigen. The peptide-cargo complexes of the present invention preferably are administered to a wide variety of cell types in vivo, in vitro, and ex vivo, including, epithelial cells, tumor cells, hepatocytes, endothelial cells, neurons, muscle cells, T-cells, dentritic cells, β-cells, primary cells, differentiated cells, stem cells, antigen presenting cells, mucosa etc. by methods known to those skilled in the art.

When administered to stem cells, such as hematopoietic cells, muscle, brain, etc., the peptide-cargo complexes of the present invention can induce differentiation of the stem cells. The peptide-cargo complex comprises factors which can stimulate differentiation of stem cells, such as the transcription factor MyoD. Stem cells isolated from bone marrow have been shown to differentiate into a wide variety of tissues, including cartilage and bone, and may be useful therapeutically (Pittenger et al. Science (1999) 284:143).

In another preferred -embodiment the peptide-cargo complex is used to expand a stemcell population. The internalising peptides of the present invention can deliver proteins to CD34+ hematopoietic progenitor stem cells. The delivery of immortalizing proteins, such as SV40 T-antigen, HPV E6, HPV E7 and telomerase, can facilitate the transient expansion of stem cell populations.

Since the delivery of the immortalizing proteins using the peptides of the present invention is transient and reversible, for example, delivery of the immortalizing protein which will be degraded subsequently in the cell, such delivery offers an advantage in that the stem cell status may be maintained, i.e. the cells may be transiently immortalized, while increasing the number of cell doublings that may be achieved. Stable delivery of immortalizing factors may also be achieved by the delivery of cargo encoding the immortalizing factor, for example a viral vector, plasmid, or DNA. In a preferred embodiment this approach is used to expand a wide variety of stem cells in culture for transplant applications.

In another preferred embodiment the peptides of the present invention are used for expanding differentiated cells, for example neurons, chondrocytes, etc., which mostly have a finite number of cell doublings in culture. The proliferation of differentiated cells may be induced by delivering immortalizing factors, preferably SV40 T-antigen, HPV E6, HPV E7, or telomerase.

It is also an object of the present invention to provide a construct comprising a peptide of the present invention linked to an antigen which can be taken up efficiently by a number of antigen presenting cells, such as dendritic cells, both in vivo and in vitro and stimulate an immune response. The peptides may be linked or complexed to viral antigens such as adeno-associated virus (AAV) Rep protein, SIV antigens or HIV antigens such as gag, pol, env, HPV-E6, HPV-E7, EBV-LMP1, EBV-LMP2, EBNA1, EBNA3A, EBNA3C, etc., ovalbumin, differentiation antigens such as MART-1/Melan A, gplOO, tyrosinase, TRP-1, TRP-2, etc., tumor specific multilineage antigens, such as MAGE-1, MAGE-3, BAGE, GAGE-1, GAGE-2, pl5, etc., antigens expressed uniquely by an individual's tumor such as mutated gene products like p53, CDK4, pl6, p21, etc.

In a preferred aspect of the invention, the immune response against tumors may be augmented by co-administration of the peptides of the present invention linked to a cargo, for example an apoptosis factor, in form of a protein-cargo complex together with, for example, cytokines and/or other activating molecules. In a preferred embodiment the cytokines and other activating molecules may be administered to the cells via the peptides of the present invention, but also by any other conventional means of administration.

Additional potential applications of the peptides of the present invention, when linked to a cargo, in particular in form of the peptide-cargo complex of the present invention, include the treatment of accessible head and neck tumors, papylomas, and other solid tumors, with the cargo comprising an apoptosis factor. In a preferred embodiment the peptide-cargo complex of the present invention is used to act as an adjuvant therapy in conjunction with radio therapy, standard chemotherapy or for surgical debulking to extend excision margins.

The present invention is further directed to promoting the growth of defective viruses, such as HSV, in culture. The generation of defective viruses is useful for gene therapy applications. Defective viruses do not replicate without the help of necessary replication proteins, such as ICPO, ICP4, ICP22 and ICP27, which are not encoded by such defective viruses. In one embodiment of the present invention, replication defective virus is grown in cells by infecting the cells with the defective virus and administering one or more complexes comprising a peptide of the present invention linked to a protein necessary for the replication of the defective virus.

In another preferred embodiment according to a further aspect of the invention metals as cargo are preferably transported into and across one or more layers of epithelial and endothelial tissues using chelating agents such as texaphyrin or diethylene triamine pentacetic acid (DTPA), and the delivery protein. These combinations are useful for delivering metal ions for imaging or therapy. Exemplary, cargo include metal ions such as Eu, Lu, Pr, Gd, ^{99m}TC, ⁶⁷Ga, ¹¹¹In, ⁹⁰Y, ⁶⁷Cu, and ⁵⁷Co. In a preferred embodiment, the metal is incorporated into the delivery protein itself or combined as cargo with the protein.

In still another preferred embodiment according to a further aspect of the invention boron reagents as cargo such as those used in Boron Neutron Capture therapy are included. In a preferred embodiment, the boron species is incorporated into the delivery protein itself or is combined as cargo with the protein.

In another preferred embodiment the cargo is an anti-aging agent selected from the group consisting of retinoic acid, hyaluronic acid, collagen, and free radical catchers, in particular SOD.

In another preferred embodiment the cargo is a therapeutic used for ulcers, preferably selected from the group consisting of an H2-histamine inhibitor, an inhibitor of the proton-potassium ATPase, and an antibiotic directed at Helicobacter pylori.

In another preferred embodiment the cargo is a therapeutic agent used for treating a bronchial condition, preferably selected from the group consisting of cystic fibrosis, asthma, allergic rhinitis, and chronic obstructive pulmonary disease.

In another preferred embodiment the cargo is a therapeutic agent used for treating ischemia, Parkinson's disease, schizophrenia, cancer, acquired immune deficiency syndrome (AIDS), infections of the central nervous system, epilepsy, multiple sclerosis, neurodegenerative disease, trauma, depression, Alzheimer's disease, migraine, pain, and a seizure disorder.

In another preferred embodiment the cargo is a therapeutic agent used for treating glucocerebrosidase deficiency (Gaucher's disease), mucopolysaccharidosis I, sanfilippo B syndrome, pancreatic insufficiency, severe combined immunodeficiency syndrome, and neuromuscular dysfunction associated with triose phosphate isomerase deficiency.

The technical problem is further solved by the use of the peptide for cellular internalisation of a cargo molecule linked thereto as well as by the use of the peptide for the production of a kit for cellular internalisation of a cargo molecule linked thereto in an animal or a human body.

The technical problem is further solved by the use of the peptide for nuclear translocation in a target cell as well as by the use of the peptide for the production of a kit for nuclear translocation in a target cell in an animal or a human body.

The technical problem is further solved by the use of the peptide for translocation in the mitochondria of a target cell as well as by the use of the peptide for the production of a kit for translocation in the mitochondria of a target cell in an animal or a human body.

The technical problem is further solved by the use of the peptide for the treatment of Crohn's disease, ulcerative colitis, gastrointestinal ulcers, peptic ulcer disease, abnormal proliferative diseases, an infection with Helicobacter pylori, cystic fibrosis, asthma, allergic rhinitis, chronic obstructive pulmonary disease, ischemia, Parkinson's disease, schizophrenia, cancer, acquired immune deficiency syndrome (AIDS), infections of the central nervous system, epilepsy, multiple sclerosis, neurodegenerative disease, trauma, depression, Alzheimer's disease, migraine, pain, and a seizure disorder, as well as by the use of a composition, in particular a pharmaceutical composition or medicament for the treatment of said diseases in an animal or a human body.

The technical problem is further solved by the provision of a pharmaceutical composition comprising:
a cargo-peptide complex, comprising an effective amount of a biologically active or therapeutic agent, and
a pharmaceutically acceptable carrier.

The technical problem is further solved by the provision of a method for the delivery of a cargo to the surface of, into or across a biological barrier, comprising the steps of:
a) providing a cargo and at least one delivery peptide according to the invention,
b) forming a peptide-cargo complex,
c) contacting the barrier with the peptide-cargo complex, and
d) delivering the cargo to the surface of, into or across the barrier.

In a preferred embodiment the barrier is an intact epithelial or endothelial tissue layer or layers. Preferably, the barrier is the skin. Preferably, the cargo is delivered into and/or across one or more of the layers stratum corneum, stratum granulosum, stratum lucidum, and stratum germinativum. In a preferred embodiment the contacting of the skin with the peptide-cargo complex is accomplished by administering a composition comprising the peptide-cargo complex topically to the skin, and in particular the cargo is taken up by cells that comprise the follicular or interfollicular epidermis. Preferably, the composition is a cream, ointment, salve, lotion, or a transdermal patch.

In another preferred embodiment the barrier is the gastrointestinal tract. In another preferred embodiment the barrier is the pulmonary epithelium. In another preferred embodiment the barrier is the endothelial blood brain barrier.

The technical problem is further solved by the provision of a method for inducing synovial cell death comprising administering the peptide-cargo complex to said synovial cell as well as by the use of the peptide-cargo complex for the production of a composition for inducing synovial cell death in an animal or a human body.

A further embodiment of the invention relates to a method for inducing apoptosis in a tumour cell comprising administering the peptide-cargo complex, in particular comprising an apoptotic protein, to said tumour cell as well as by the use of the peptide-cargo complex, in particular comprising an apoptotic protein, for the production of a composition for inducing apoptosis in a tumour cell in an animal or a human body.

A further embodiment of the invention relates to a method for reducing white blood cells in arthritic joints comprising administering the peptide-cargo complex to said white blood cells as well as by the use of the peptide-cargo complex for the production of a composition for reducing white blood cells in arthritic joints in an animal or a human body.

A further embodiment of the invention relates to a method for reducing the effects of skin aging comprising administering the peptide-cargo complex, in particular comprising an anti-aging agent, to the skin as well as by the use of the peptide-cargo complex, in particular comprising an anti-aging agent, for the production of a composition for reducing the effects of skin aging in an animal or a human body. Preferably, the composition forms or comprises a cream, ointment, salve, or lotion.

A further embodiment of the invention relates to a method for eliciting an immune response in an animal or a human body comprising administering to a target cell of said body an immunogen comprising the peptide-cargo complex as wells as by the use of the peptide-cargo complex for the production of a immunogen for eliciting an immune response in an animal or a human body.

Still another aspect of the invention relates to fusion proteins. For example GST fusion proteins are widely used in research to study various proteins due to the ease of expressing and purifying such fusion proteins. In one embodiment of the present invention the peptides of the present invention are used for delivering one or more GST fusion protein to cells. The peptides of the present invention, preferably when linked to glutathione, facilitate the delivery of GST fusion proteins in a target cell. Such preferred glutathione-peptide constructs bind to any GST fusion protein and facilitate the internalisation of the GST fusion protein into a cell.

A further embodiment of the invention is an expression cassette comprising a nucleic acid encoding a fusion protein comprising a leader sequence, an internalising peptide of the present invention, and a protein of interest, operably linked to expression control sequences. Such a fusion protein is capable of post-translational intercellular transport via the leader sequence or the internalising peptides of the present invention. The leader sequence is preferably derived from secreted gene products such as interleukin-1 receptor antagonist (IL-1ra), Parathyroid hormone (PTH), or cathelin (Huttner et al., Ped. Res. (1999) 45:785). Preferably, the leader sequence is clipped or removed during translocation. Preferably, the peptides of the invention ensure that the fusion protein encoded by the expression cassette of the present invention is internalised into surrounding cells, even after removal of the leader sequences, thereby improving the efficiency of intercellular transport. The cargo comprising the protein of interest may include, apoptotic proteins, suicide proteins, therapeutic proteins, etc.

In addition, VP22 protein of herpes simplex virus has been shown to be released from cells and taken up by neighbouring cells. Accordingly, another preferred embodiment of the present invention is directed to a fusion construct comprising the leader sequence of VP22, a peptide of the present invention, and a cargo, for example an apoptotic protein, suicide protein, therapeutic protein, etc.

The embodied expression cassette comprises a DNA transgene encoding a fusion protein comprising a leader sequence, a protein of interest and the delivery peptide. In a preferred embodiment the expression cassette further comprises expression control sequences operatively linked to said DNA. In a preferred embodiment the expression cassette of the present invention preferably is contained within a transfer vector which preferably is administered to cells either in vivo or in vitro and mediate expression therein. The expression cassette comprising DNA sequences encoding a fusion protein comprising a leader sequence, an internalising peptide and a protein of interest is useful to direct the delivery of the protein of interest to surrounding cells. In a preferred embodiment the protein of interest is an apoptotic protein, anti-apoptotic protein, cell cycle regulatory protein, transcription factor, suicide gene product, viral or tumor antigens, or cell proliferation factors, for example viral oncoproteins, telomerase, etc.

A further embodiment of the invention relates to a transfer vector comprising the expression cassette. In addition to containing the DNA sequences encoding one or more transgenes, preferred chimeric viral vectors, in particular adenoviral vectors, preferably contain expression control sequences such as a promoter or enhancer, a polyadenylation element, and any other regulatory element used to modulate or increase expression. Preferably, all are operably linked in order to allow expression of the transgene. The use of any expression control sequences, or regulatory elements, which facilitate expression of the transgene is within the scope of the invention. Such sequences or elements preferably are capable of generating tissue-specific expression or be susceptible to induction preferably by exogenous agents or stimuli. For example, suitable promoters include promoters such as from phosphoglycerate kinase (PGK) promoter or a cytomegalovirus (CMV).

In one preferred embodiment, a vector containing the expression cassette comprising DNA sequences encoding a fusion protein comprising a leader sequence, a peptide of the invention and cargo, preferably a protein of interest, is administered to a cell wherein said expression cassette is preferably transcribed and translated and the resultant fusion protein preferably is then secreted via the leader sequences. After secretion from the cell in which it was expressed, the fusion protein comprising the delivery peptide of the invention, a therapeutic protein or other protein of interest, and optionally the leader sequence preferably is internalised into surrounding cells in vivo or in vitro via the delivery peptides of the present invention.

The embodied expression cassette is useful for sustained delivery of a peptide-cargo complex in cells. Any leader sequence capable of directing the secretion of a polypeptide linked thereto is contemplated by the present invention, including, but not limited to IL-1ra, PTH and related sequences.

The invention is also related to methods of identifying delivery peptides of the present invention. Peptides having the ability to be internalised into cells can be identified by random peptide libraries coupled with an affinity enrichment process. Preferably, a phage display peptide library kit is employed for the identification of delivery peptides which are capable of being internalised into cells and are also capable of facilitating the internalisation of cargo into cells. Preferably, a random peptide library is presented on a plasmid, in particular as part of a fusion protein or protein as a peptide-protein complex by techniques known in the art. Methods of identifying delivery peptides facilitate the isolation of peptides with superior internalising capabilities and provide numerous peptides which can be selected for a reduced likelihood of eliciting an immune response when administered to a subject and an increased half life in vivo and/or in vitro. The method preferably comprises incubating a target cell with a peptide display library, isolating internalised peptide presented by said peptide display library from the cytoplasm and nuclei of the cells and identifying said peptides.

The sequence listing includes:

SEQ ID NO: 1 to SEQ ID NO: 39: amino acid sequences of artificial oligopeptides according to the present invention in particular functioning as delivery peptides; SEQ ID NO: 40 and SEQ ID NO: 41: control peptides.

Although only preferred embodiments of the invention are specifically described above and in the following examples, it will be appreciated that the embodiments and examples are provided to more clearly illustrate the aspects of the invention and are not intended to limit the scope of the present invention. Further modifications and variations of the invention are possible within the scope of invention.

The figures show:

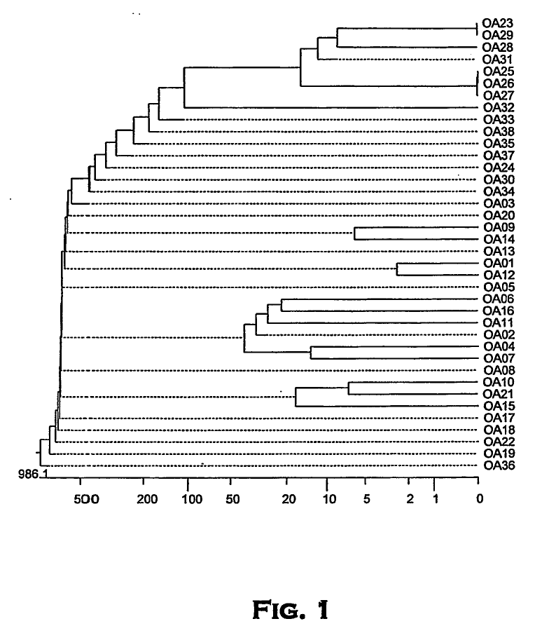

FIG. 1 depicts a similarity analysis of the embodied peptide family. The newly designed peptide sequences (see example 1 for the design-rationale) were analysed using DNASTAR Megalign® (mode: slow/accurate with general Blosum tables). Within the family the sequences are structurally highly divergent.

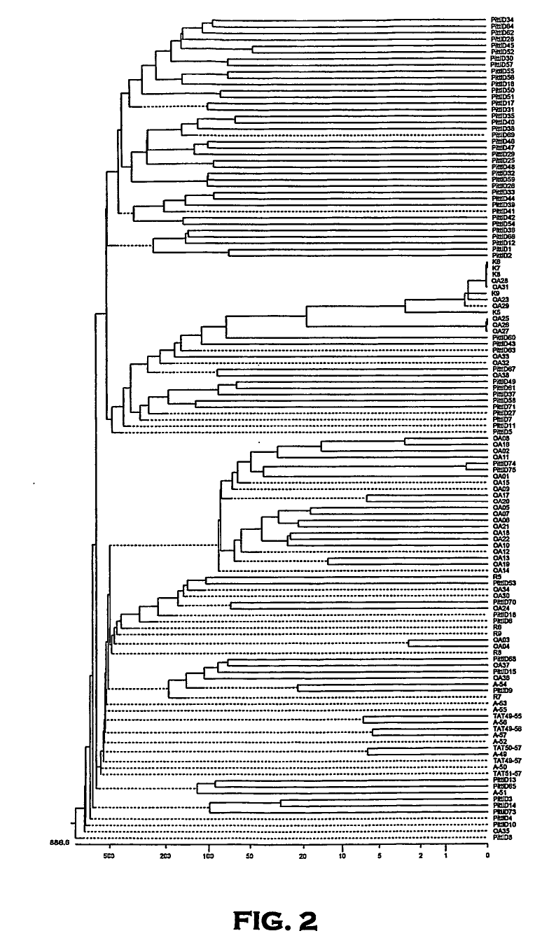

FIG. 2 depicts a similarity analysis of the embodied peptide family and known peptide PTD families. Published PTDs (WO 91/09958, WO 02/069930) and newly designed peptide sequences (see example 1) were analysed using DNASTAR Megalign® (mode: slow/accurate with general Blosum tables). Compared to known peptides, the new peptide family forms a distinct group, of which some members have a slight similarity to known peptides.

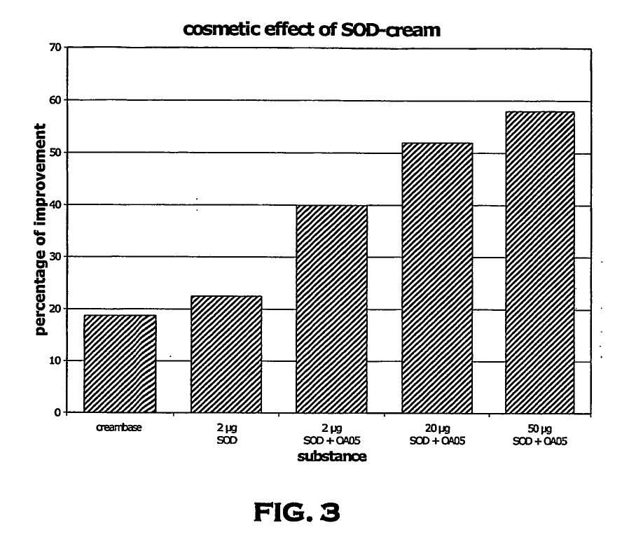

FIG. 3 shows a bar graph of the evaluation of the cosmetic effect of the huSOD-peptide-OA 05-conjugate in a cream base. Shown are the percentages of improved skin care parameters as a function of the different treatment groups. Improvement is increased as the concentration of huSOD and OA 05 increases.

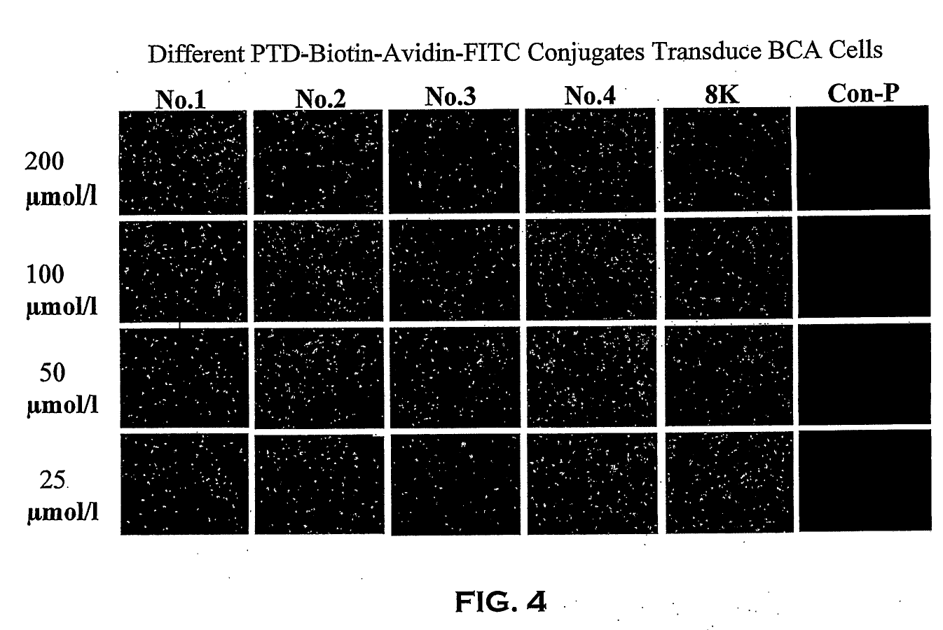

FIG. 4 shows that different PTD's-biotin-avidin-FITC-conjugates transduce BCA-cells.

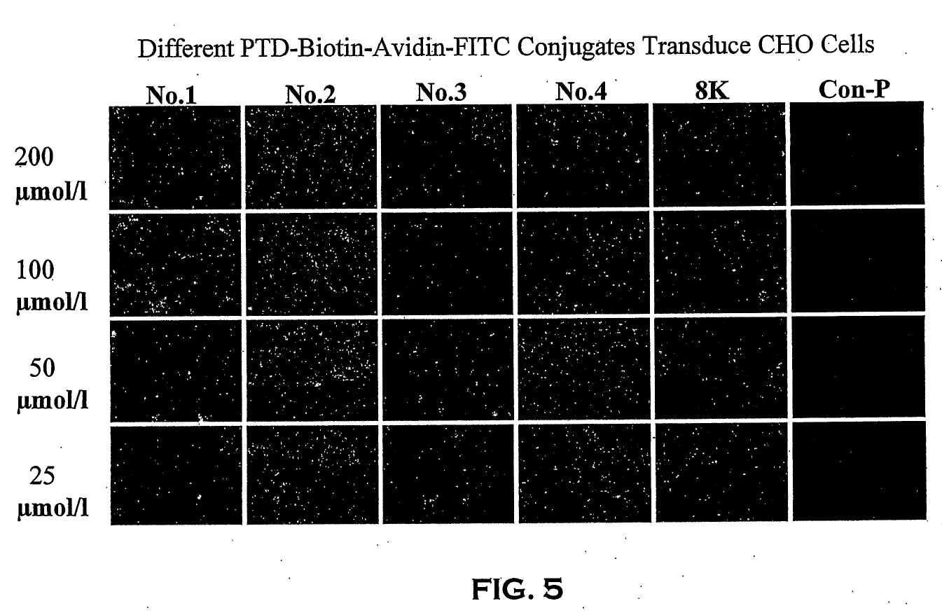

FIG. 5 shows that different PTD-biotin-avidin-FITC-conjugates transduce CHO-cells.

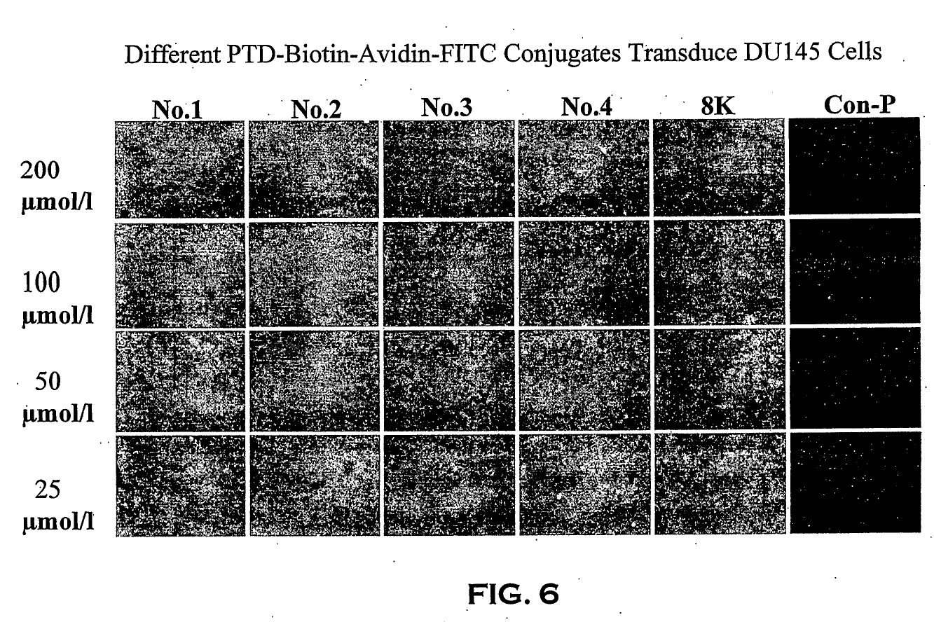

FIG. 6 shows that different PTD-biotin-avidin-FITC-conjugates transduce DU145 cells.

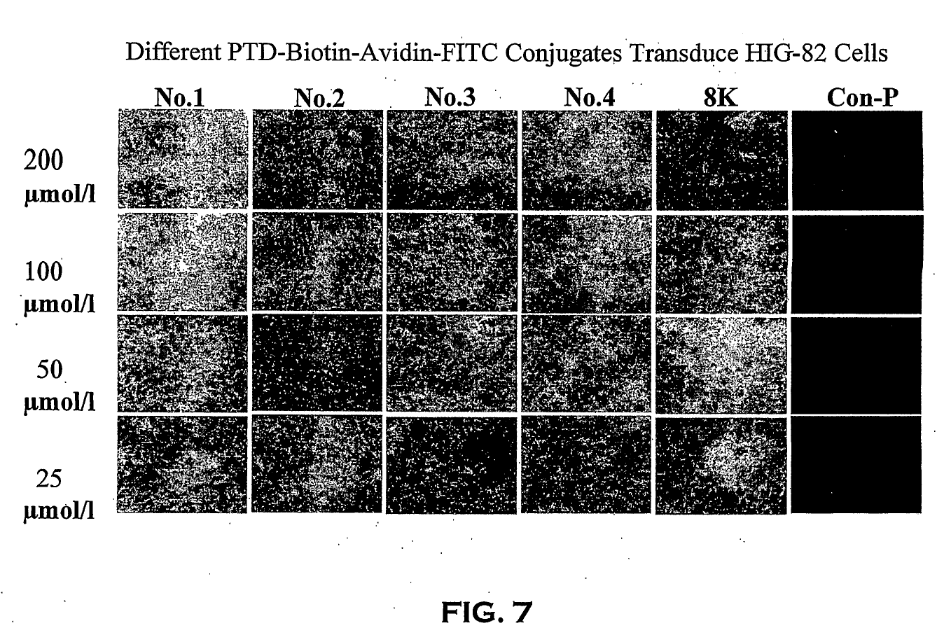

FIG. 7 shows that different PTD-biotin-avidin-FITC-conjugates transduce HIG-82 cells.

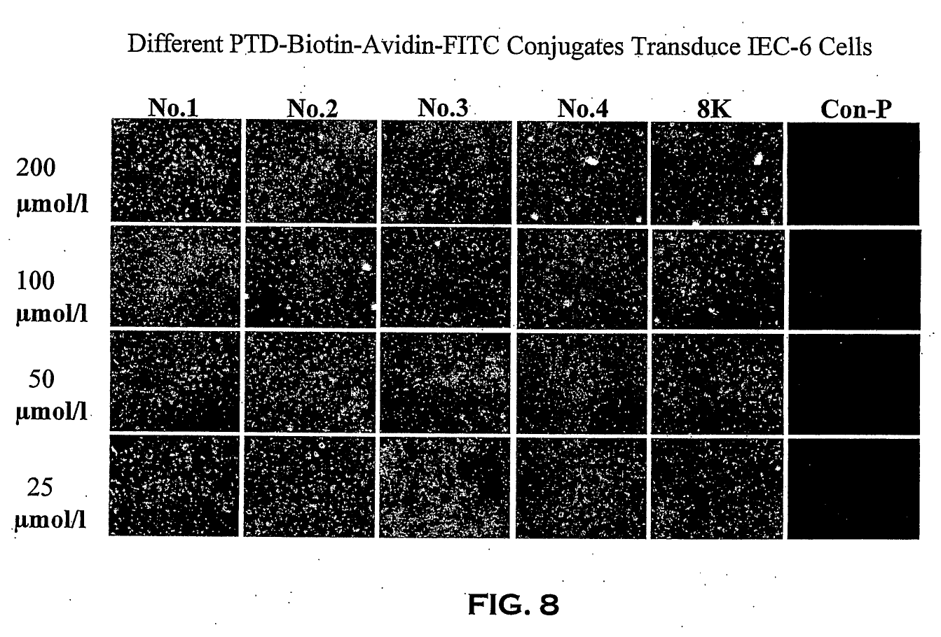

FIG. 8 shows that different PTD-biotin-avidin-FITC-conjugates transduce IEC-6-cells.

### EXAMPLE 1

### In Vitro Translocation of Biotinylated Peptides into Fibroblasts

Biotinylated peptides were synthesized by a commercial supplier (Jerini AG, Berlin). The biotin amount was 5 mg, purity was higher than 95% (HPLC, 220 nm, C18 and C4, linear gradient, analyses: HPLC & MS, with counter ion: chloride).

The following peptides were synthesised:

I. Known Peptides

Group 1 (comparative examples taken
from WO 02/069930)

| | |
|---|---|
| CE 49 | AKKRRQRRR |
| CE 50 | RAKRRQRRR |
| CE 51 | RKARRQRRR |
| CE 52 | RKKARQRRR |

Group 2 (comparative examples taken
from WO 01/15511)

| | |
|---|---|
| PI 01 | KRIIQRILSRNS |
| PI 02 | KRIHPRLTRSIR |
| PI 03 | PPRLRKRRQLNM |
| PI 04 | PIRRRKKLRRLK |
| PI 05 | RRQRRTSKLMKR |

II. Peptides According to the Invention

Peptides according to the general formula I
(K)ₙA₁B₁C₁(K)ₘA₂B₂C₂(K)ₗA₃B₃C₃(K)ₒ, (I)
wherein
K is lysine (K),
n, m, l, o is an integer from 0 to 5,
B₁, B₂, B₃ is arginine (R), glutamine (Q) or histidine (H),
A₁, A₂, A₃, C₁, C₂, C₃ is arginine (R), histidine (H) or is missing, and the total number of amino acid residues is not more than 10

were synthesised:

| | |
|---|---|
| KKRKKQKKRK, | (SEQ ID NO:1) |
| RRKKKQKKK, | (SEQ ID NO:2) |
| KKQKKRRK, | (SEQ ID NO:3) |
| KKQKKRRKK, | (SEQ ID NO:4) |
| KKKQKRKK, | (SEQ ID NO:5) |
| RQKKQKKKR, | (SEQ ID NO:6) |
| RKQKKKRKKK, | (SEQ ID NO:7) |
| KRKQKQKKK, | (SEQ ID NO:8) |
| KKRKQKKQK, | (SEQ ID NO:9) |
| KKKRKKQK, | (SEQ ID NO:10) |
| RKKKKQKKK, | (SEQ ID NO:11) |
| KKRKKQKK, | (SEQ ID NO:12) |
| QKKRRKKKQK, | (SEQ ID NO:13) |
| KKRKQKKRK, | (SEQ ID NO:14) |
| KKRKQKKQKR, | (SEQ ID NO:15) |
| KRKQKQKKKK, | (SEQ ID NO:16) |
| KKRKRKQKK, | (SEQ ID NO:17) |
| KQKRKKKQK, | (SEQ ID NO:18) |
| KQKKRQKKKR, | (SEQ ID NO:19) |
| KKKRKQKQKK, | (SEQ ID NO:20) |
| RKKKQKKQKK, | (SEQ ID NO:21) |
| KKKRQKKQK, | (SEQ ID NO:22) |
| KKRKKKKKRK, | (SEQ ID NO:23) |
| RRKKKKKK, | (SEQ ID NO:24) |
| KKKKRRK, | (SEQ ID NO:25) |
| KKKKRRKK, | (SEQ ID NO:26) |
| KKKKRKK, | (SEQ ID NO:27) |
| KKRKKKKK, | (SEQ ID NO:28) |
| KKRKKHKKRK, | (SEQ ID NO:29) |
| RRKKKHKKK, | (SEQ ID NO:30) |
| KKHKKRRK, | (SEQ ID NO:31) |
| KKHKKRRKK, | (SEQ ID NO:32) |
| KKKHKRKK, | (SEQ ID NO:33) |
| RHKKHKKKR, | (SEQ ID NO:34) |
| RKHKKKRKKK, | (SEQ ID NO:35) |
| HHKRKKKRK, | (SEQ ID NO:36) |
| KKRHHKRK, | (SEQ ID NO:37) |
| KKHRKKH, | (SEQ ID NO:38) |
| and | |
| KKKQKRK. | (SEQ ID NO:39) |

Accordingly, the synthesised peptides according to the invention can be divided in three subgroups: (a) SEQ ID NO: 1 to SEQ ID NO: 22 and SEQ ID NO: 39: peptides consisting of lysine (K), arginine (R) and glutamine (Q); (b) SEQ ID NO: 23 to SEQ ID NO: 28: peptides consisting of lysine (K) and arginine (R); and (c) SEQ ID NO: 29 to SEQ ID NO: 38: peptides consisting of lysine (K), arginine (R) and histidine (H).

For the design of subgroup (a) of peptides according to the invention the relative number of lysine (K) residues in comparison to known proteins was increased, basic charge was obtained by the incorporation of one or a maximum of two arginine (R) residues.

For the design of subgroup (b) of peptides according to the invention one or a maximum of two residues of histidine (H) were incorporated, the peptides obtained have a high basic charge at low pH. For the following examples peptides were selected from the above peptides according to the invention and grouped in group 3, representing subgroup (a), and group 4, representing subgroup (b) of the peptides according to the invention.

Group 3 (according to the invention)

| | | |
|---|---|---|
| OA 01 | KKRKKQKKRK | (SEQ ID NO:1) |
| OA 02 | RRKKKQKKK | (SEQ ID NO:2) |
| OA 03 | KKQKKRRK | (SEQ ID NO:3) |
| OA 04 | KKQKKRRKK | (SEQ ID NO:4) |
| OA 05 | KKKQKRKK | (SEQ ID NO:5) |
| OA 39 | KKKQKRK | (SEQ ID NO:39) |

Group 4 (according to the invention)

| | | |
|---|---|---|
| OA 34 | RHKKHKKKR | (SEQ ID NO:34) |
| OA 35 | RKHKKKRKKK | (SEQ ID NO:35) |
| OA 36 | HHKRKKKRK | (SEQ ID NO:36) |
| OA 37 | KKRHHKRK | (SEQ ID NO:37) |
| OA 38 | KKHRKKH | (SEQ ID NO:38) |

Varying concentrations (0.1 mmol/l to 1 mol/l of final concentration) of each peptide with biotin covalently attached to the amino terminus dissolved in phosphate buffered saline (PBS), were applied to confluent adherent NIH/3T3 fibroblast cultures in flask slides (from Nunc). The cell cultures were incubated with the peptide for thirty minutes. Cells were fixated with formaldehyde (2% formaldehyde, 0.2% glutaraldehyde in 1×PBS) and lysed with 0.05% trypsin in 0.5 mmol/l EDTA. The slides were stained by incubation with fluorescence-labelled streptavidin and were analysed by fluorescent microscopy.

Results:

The cells took up each tested peptide. An increase in peptide concentration leads to an increase in fluorescent labelling (as measured by the percentage of labelled cells). No significant difference in uptake of the different peptides (as measured by the percentage of labelled cells) was observed. This demonstrates that the peptides can deliver in vitro conjugated biotin into murine fibroblasts.

### EXAMPLE 2

### In Vitro Translocation of Biotinylated Peptides into Fibroblasts Depends on the pH

Varying concentrations (0.1 mmol/l to 1 mol/l of final concentration) of each peptide from group 3 and group 4 (see example 1) with biotin covalently attached to the amino terminus dissolved in TRIS buffered saline (either at pH 6.0, 7.0 or 8.0), were applied to confluent adherent NIH/3T3 fibroblast cultures in flask slides (from Nunc). The cell cultures were incubated with the peptide for thirty minutes. Cells were fixated with formaldehyde (2% formaldehyde, 0.2% glutaraldehyde in 1×PBS) and lysed with 0.05% trypsin in 0.5 mmol/l EDTA. The slides were stained by incubation with fluorescence-labelled streptavidin and were analysed by fluorescent microscopy.

Results:

At pH 6.0 the cells took up each tested peptides. At pH 7.0 and 8.0 the uptake of group 4 peptides (as measured by the percentage of labelled cells) was reduced in comparison to the uptake of group 3 peptides. This demonstrates that the delivery in vitro of conjugated biotin by peptides into murine fibroblasts depends on the pH and that histidine only contributes to translocation at low pH.

### EXAMPLE 3

### Specific Activity of Peptide-Enzyme Complexes

All peptides of example 1 were coupled using disulfide bridges to various epitopes of Lac Z and to epitopes of GFP. After coupling and purification the specific biological activity of the peptide-enzyme complex was determined. Lac Z activity was determined using Beta-Gal assays or staining kits (from Invitrogen). Specific activity was determined either based on total protein content (Bradford, Biorad) or on cell numbers. Steric hindrance was estimated by relating detected epitope (GFP-antiserum, Invitrogen) to the total GFP amount.

Results:

Masking of the epitopes was not significantly different between the peptide groups. The specific activity of enzymes coupled to group 1 or to group 2 peptides was significantly reduced. However, the specific activity of enzymes coupled to group 3 or group 4 peptides was not significantly reduced. This shows that the effect of peptides, coupled to proteins, on the biological specific activity of these proteins depends on the sequence of the coupled peptide.

### EXAMPLE 4

### Specific Activity of Peptide-huSOD Complexes

All peptides of example 1 were coupled to various epitopes of huSOD (from Jena Biosciences) using disulfide bridges. After coupling and purification the specific biological activity of the peptide-enzyme complex was determined by using the Superoxide Dismutase Assay Kit (from Calbiochem) and the measurement of total protein content (from Bradford, Biorad). Steric hindrance was estimated by relating detected epitope (anti-SOD, from abcam) to the total SOD amount.

Results:

Masking of the epitopes was not significantly different between the peptide groups. The specific activity of huSOD coupled to group 1 or group 2 peptides was significantly reduced. However, the specific activity of enzymes coupled to group 3 or group 4 peptides was not significantly reduced. This shows that the effect of peptides, coupled to proteins, on the biological specific activity of these proteins depends on the sequence of the coupled peptide.

### EXAMPLE 5

### Penetration of Peptides into the Skin of Nude Mice

Varying concentrations, 0.1 mmol/l to 1 mol/l, of the peptides CE 49, PI 05, OA 01, OA 05, and OA 34 with biotin covalently attached to the amino terminus dissolved in phosphate buffered saline (PBS), were applied to the back of anaesthetized nude mice. Samples were allowed to penetrate for thirty minutes. Subsequently the animal was sacrificed, the relevant sections of skin were excised, embedded in mounting medium and frozen. Frozen sections (5 microns) were stained with fluorescence-labelled streptavidin. Slides were analysed by fluorescent microscopy.

Results:

The conjugated biotin was transported into and across the epidermis and into the dermis by all peptides. No significant difference in translocation could be observed. This demonstrates that the peptides can deliver conjugated biotin into skin.

### EXAMPLE 6

### Penetration of huSOD into the Skin of Nude Mice

This example demonstrates that peptides can deliver conjugated huSOD into skin and that the activity of huSOD depends on the sequence of the peptide. The specific activity of huSOD coupled to group 1 or group 2 peptides as determined by the number of stained cells was significantly different in comparison to the activity of huSOD coupled to group 3 or group 4 peptides.

Varying concentrations of 1 mmol/l to 1 mol/l of the peptides CE 49, PI 05, OA 01, OA 05, and OA 34 with huSOD covalently attached to the amino terminus dissolved in phosphate buffered saline (PBS), were applied to the back of anaesthetised nude mice. Samples were allowed to penetrate for thirty minutes. Subsequently the animal was sacrificed, the relevant sections of skin were excised, embedded in mounting medium and frozen. Fixated sections (5 microns) were either stained with a fluorescence-labelled anti-huSOD antibody (abcam) or an in situ SOD-assay (from Calbiochem) was performed. Slides were analysed by microscopy.

Results:

The conjugated huSOD was transported into and across the epidermis and into the dermis by all peptides. With huSOD conjugated with proteins of group 1 and group 2 no significant difference in translocation could be observed after staining with the anti-huSOD antibody. In contrast, huSOD conjugated with proteins of group 3 and group 4 was transported into and across the epidermis and into the dermis at a considerably higher rate, as significantly increased staining with the anti-huSOD antibody was observed.

### EXAMPLE 7

### Similarity Analysis of Peptide Motives

Published PTDs (see WO 91/09958 or WO 02/069930) and delivery peptides with newly designed sequences (see example 1) were analysed using DNASTAR Megalign® (mode: slow/accurate with general Blosum tables).

Results:

The analyses (FIG. 1 and FIG. 2) demonstrate that the embodied peptide represent a new structural family of PTDs.

### EXAMPLE 8

### Clinical Study with a Cream for Skincare Against Skin Aging

A cream base was used having the following ingredients:

| substance: | amount [g] |
|---|---|
| tocopherol acetate | 10.0 |
| essence of calendula | 10.0 |
| lecithine | 5.0 |
| DMS (emulsifying agent) | 2.0 |
| glycerine | 10.0 |
| shea butter | 30.0 |
| Cordes creme base | 10.0 |
| aqua dest. | ad 100.0 |

The huSOD-peptide OA 05 conjugate was produced as described in example 4.

The following compositions of skin cream were prepared for use in the clinical study:

| | |
|---|---|
| Group I (control): | cream base without SOD (n = 20) |
| Group II (control): | cream base with 2 μg SOD per 1 g |
| | of skin cream (n = 20) |
| Group III | cream base with 2 μg SOD + peptide |
| (according to the | OA 05 per 1 g of skin |
| invention): | cream (n = 22) |
| Group IV | cream base + 20 μg SOD + peptide |
| (according to the | OA 05 per 1 g of skin |
| invention): | cream (n = 15) |
| Group V | cream base + 50 μg SOD + peptide |
| (according to the | OA 05 per 1 g of skin |
| invention): | cream (n = 10) |

Cosmetic evaluation properties were evaluated with a prospective self evaluation standardized evaluation form with evaluation scores ranging from “completely agree”; “agree” to “do not agree” and with a further category “no opinion”. The properties were evaluated according the following criteria: “firmness of the skin”, “reduction of superficial wrinkles”, “reduction of deeper wrinkles”, “skin regeneration”, and “appearance of young skin”. The questionnaire was filled out at least 4 weeks after starting of using the skin cream.

The application of the skin cream was done in all Groups (I to V) over 6 weeks twice a day, creaming the face and the neck in the morning and in the evening without using any other skin products. No application was discontinued in any group. The observation period (counted from the first application) was minimum 4 weeks and maximum 10 weeks.

Furthermore, a total evaluation of the different applied skin creams on a score ranging from 1 (excellent) to 6 (very poor) was performed.

Results:

All 87 participants answered questions oriented towards a standardised score concerning side effects, cosmetic characteristics, reduction of wrinkles, etc.

Table 1 lists different cosmetic evaluation properties as a function of treatment modality.

TABLE 1

| | evaluation | | | |
|---|---|---|---|---|
| | | | do | |
| | | | not | no |
| treatment | completly | agree | agree | opinion |
| creme base (n = 20) | | | | |
| firmer skin | 3 | 5 | 7 | 5 |
| superficial wrinkles reduced | 3 | 7 | 5 | 5 |
| deeper wrinkles reduced | 2 | 5 | 7 | 5 |
| skin regenerated | 7 | 7 | 3 | 3 |
| skin appears younger | 4 | 5 | 7 | 4 |
| creme base + 2 μg SOD | | | | |
| (n = 20) | | | | |
| firmer skin | 4 | 6 | 9 | 1 |
| superficial wrinkles reduced | 3 | 5 | 8 | 4 |
| deeper wrinkles reduced | 3 | 8 | 6 | 3 |
| skin regenerated | 7 | 8 | 2 | 3 |
| skin appears younger | 5 | 7 | 5 | 3 |
| creme base + 2 μg SOD + OA05 | | | | |
| (n = 22) | | | | |
| firmer skin | 10 | 8 | 1 | 3 |
| superficial wrinkles reduced | 8 | 12 | 0 | 2 |
| deeper wrinkles reduced | 6 | 11 | 1 | 4 |
| skin regenerated | 10 | 12 | 0 | 0 |
| skin appears younger | 10 | 9 | 0 | 3 |
| creme base + 20 μg SOD + OA05 | | | | |
| (n = 15) | | | | |
| firmer skin | 9 | 4 | 0 | 2 |
| superficial wrinkles reduced | 8 | 5 | 2 | 0 |
| deeper wrinkles reduced | 6 | 8 | 1 | 0 |
| skin regenerated | 9 | 6 | 0 | 0 |
| skin appears younger | 7 | 7 | 1 | 0 |
| creme base + 50 μg SOD + OA05 | | | | |
| (n = 10) | | | | |
| firmer skin | 5 | 5 | 0 | 0 |
| superficial wrinkles reduced | 5 | 3 | 1 | 1 |
| deeper wrinkles reduced | 6 | 2 | 1 | 1 |
| skin regenerated | 7 | 2 | 0 | 1 |
| skin appears younger | 6 | 3 | 1 | 0 |

Table 2 lists the results of the total evaluation score from 1 (excellent) to 6 (very poor) of the different applied skin creams in humans. The scores 5 and 6 were not rated in any of the groups. Note the great difference in the evaluation between the experimental groups (huSOD+OA 05) and the controls.

TABLE 2

| | evaluation | | | | | |
|---|---|---|---|---|---|---|
| | | | | | further | no further |
| Treatment | 1 | 2 | 3 | 4 | use | use |
| creme base (n = 20) | 4 | 8 | 7 | 1 | 15 | 5 |
| creme base + 2 μg SOD | 5 | 8 | 5 | 2 | 13 | 7 |
| (n = 20) | | | | | | |
| creme base + 2 μg SOD + OA05 | 16 | 5 | 1 | 0 | 22 | 0 |
| (n = 22) | | | | | | |
| creme base + 20 μg SOD + | 12 | 3 | 0 | 0 | 15 | 0 |
| OA05 (n = 15) | | | | | | |
| creme base + 50 μg SOD + | 8 | 2 | 0 | 0 | 9 | 1 |
| OA05 (n = 10) | | | | | | |

No side effects have been observed. In one case (Group IV) an allergy was suspected, but after continuation of the same product no further allergic reactions were observed. A follow up over 3 months did not show any recurrence.

The comparison between SOD and basic skin cream without addition of peptide-conjugates demonstrates, that SOD itself has a beneficial effect on different aging parameters in the human skin. The conjugation of peptide OA 05 however significantly increases the beneficial effect of SOD. In the peptide-cargo molecule complex of SOD and OA 05 the specific biological activity of the cargo molecule SOD is not inhibited by the attached delivery peptide OA 05.

This example shows, that the application of huSOD, a therapeutic substance, and OA 05, a group 3 peptide, is effective as an anti aging and skin care agent in humans.

### EXAMPLE 9

### Comparison of Transduction Efficiency of Different PTDs

To test transduction efficiency of a group of cationic peptides, known as protein transduction domains (PTDs), six peptides were synthesised and biotinylated. No. 1 to 4 were testing peptides, 8K was the positive control and Con-P was a random peptide, served as negative control. The peptide sequences are listed below:

| | | |
|---|---|---|
| No.1 | KKRKKQKK | (SEQ ID NO:12) |
| No.2 | KKKKRRK | (SEQ ID NO:25) |
| No.3 | KKKQKRK | (SEQ ID NO:39) |
| No.4 | KKKKRRKK | (SEQ ID NO:26) |
| 8K | KKKKKKKK | (SEQ ID NO:40) |
| Con-P | ARPLEHGSDKAT | (SEQ ID NO:41) |

The biotinylated peptides were dissolved in TBS for 200 μmol/l solution and the peptides were conjugated to FITC-labeled avidin (Sigma) at 25° C. for 2 hrs. The serial dilutions of the conjugates were added to five different sources of cells: Bovine primary chondrocytes (BCA), Chinese hamster ovary cell line (CHO), a prostate cancer cell line (DU 145), a murine intestine cell line (IEC-6), and a rabbit synovial cell line (HIG-82), and incubated at 37° C. for 3 hrs. The conditioned cells were washed with TBS extensively and the images were taken for the analysis.

As FIGS. 4 to 8 clearly show, the four peptides of the present invention show a significant transduction efficiency in all of the tested cell types.

1. A delivery peptide comprising an amino acid sequence of formula I:
(K)ₙA₁B₁C₁(K)ₘA₂B₂C₂(K)ₗA₃B₃C₃(K)ₒ, (I)
wherein
K is lysine (K),
B₁, B₂, B₃ are each selected from the group consisting of arginine (R), glutamine (Q) and histidine (H),
A₁, A₂, A₃, C₁, C₂, C₃ are each arginine (R), histidine (H) or are missing, n, m, l, o is an integer from 0 to 5, and
the total number of amino acid residues is not more than 10.

2. The peptide of claim 1 comprising an amino acid sequence selected from the group consisting of:

| | |
|---|---|
| KKRKKQKKRK, | (SEQ ID NO:1) |
| RRKKKQKKK, | (SEQ ID NO:2) |
| KKQKKRRK, | (SEQ ID NO:3) |
| KKQKKRRKK, | (SEQ ID NO:4) |
| KKKQKRKK, | (SEQ ID NO:5) |
| RQKKQKKKR, | (SEQ ID NO:6) |
| RKQKKKRKKK, | (SEQ ID NO:7) |
| KRKQKQKKK, | (SEQ ID NO:8) |
| KKRKQKKQK, | (SEQ ID NO:9) |
| KKKRKKQK, | (SEQ ID NO:10) |
| RKKKKQKKK, | (SEQ ID NO:11) |
| KKRKKQKK, | (SEQ ID NO:12) |
| QKKRRKKKQK, | (SEQ ID NO:13) |
| KKRKQKKRK, | (SEQ ID NO:14) |
| KKRKQKKQKR, | (SEQ ID NO:15) |
| KRKQKQKKKK, | (SEQ ID NO:16) |
| KKRKRKQKK, | (SEQ ID NO:17) |
| KQKRKKKQK, | (SEQ ID NO:18) |
| KQKKRQKKKR, | (SEQ ID NO:19) |
| KKKRKQKQKK, | (SEQ ID NO:20) |
| RKKKQKKQKK, | (SEQ ID NO:21) |
| KKKRQKKQK, | (SEQ ID NO:22) |
| KKRKKKKKRK, | (SEQ ID NO:23) |
| RRKKKKKK, | (SEQ ID NO:24) |
| KKKKRRK, | (SEQ ID NO:25) |
| KKKKRRKK, | (SEQ ID NO:26) |
| KKKKRKK, | (SEQ ID NO:27) |
| KKRKKKKK, | (SEQ ID NO:28) |
| KKRKKHKKRK, | (SEQ ID NO:29) |
| RRKKKHKKK, | (SEQ ID NO:30) |
| KKHKKRRK, | (SEQ ID NO:31) |
| KKHKKRRKK, | (SEQ ID NO:32) |
| KKKHKRKK, | (SEQ ID NO:33) |
| RHKKHKKKR, | (SEQ ID NO:34) |
| RKHKKKRKKK, | (SEQ ID NO:35) |
| HHKRKKKRK, | (SEQ ID NO:36) |
| KKRHHKRK, | (SEQ ID NO:37) |
| KKHRKKH | (SEQ ID NO:38) |
| and | |
| KKKQKRK. | (SEQ ID NO:39) |

3. The peptide according to claim 1 consisting of amino acids selected from histidine (H), lysine (K), glutamine (Q), and arginine (R).

4. An expression cassette comprising a DNA transgen encoding a fusion protein comprising a leader sequence, a protein of interest and the delivery peptide of claim 1.

5. The expression cassette of claim 4 further comprising expression control sequences operatively linked to said DNA.

6. A transfer vector comprising the expression cassette of claim 4.

7. A peptide-cargo complex comprising the delivery peptide of claim 1 and at least one cargo molecule.

8. The peptide-cargo complex of claim 7, wherein the delivery peptide is selectively linked to an outer surface of at least one cargo molecule.

9. The peptide-cargo complex of claim 8, wherein the linkage is formed by a cleavable linker.

10. The peptide-cargo complex of claim 8, wherein the linkage includes a disulfide bond.

11. The peptide-cargo complex of claim 8, wherein the linkage includes a streptavidin-biotin complex.

12. The peptide-cargo complex of claim 7, wherein the delivery peptide is biotinylated and the cargo molecule is avidin labeled.

13. The peptide-cargo complex of claim 7, wherein the cargo comprises at least one compound selected from the group consisting of polynucleotides, polypeptides, proteins, small organic molecules, metals, viruses, modified viruses, viral vectors, and plasmides.

14. The peptide-cargo complex of claim 7, wherein the cargo is a virus selected from the group consisting of adenoviruses, adeno-associated viruses, herpes viruses, simplex virus, and retroviruses.

15. The peptide-cargo complex of claim 7, wherein the cargo is selected from the group consisting of therapeutic proteins, suicide proteins, tumor suppressor proteins, transcription factors, kinase inhibitors, kinases, regulatory proteins, apoptotic proteins, anti-apoptotic proteins, viral antigens, cellular antigens, differentiation factors, immortalisation factors, toxines, enyzmes, nucleic acids, antisense constructs, diagnostic imaging or contrast agents, dyes, antibacterial agents, antifungal agents, antiviral agents, antiproliferative agents, cytostatics, immunosuppressive agents, vitamins, analgesic agents, hormones, antiinflammatory agents, and antiaging agents.

16. The peptide-cargo complex of claim 7, wherein the cargo is an antiviral agent selected from the group consisting of acyclovir, famciclovir, ganciclovir, foscarnet, idoxuridine, sorivudine, trifluridine (trifluoropyridine), valacyclovir, cidofovir, didanosine, stavudine, zalcitabine, zidovudine, ribavirin and rimantatine.

17. The peptide-cargo complex of claim 7, wherein the cargo is an antibacterial agent selected from the group consisting of nafcillin, oxacillin, penicillin, amoxacillin, ampicillin, cephalosporine, cefotaxime, ceftriaxone, rifampin, minocycline, ciprofloxacin, norfloxacin, erythromycin, tetracycline, gentamicin, a macrolide, a quinolone, a β-lactone, a P-lactamase inhibitor, salicylamide, and vancomycin.

18. The peptide-cargo complex of claim 7, wherein the cargo is an antifungal agent selected from the group consisting of amphotericin, itraconazole, ketoconazole, miconazole, nystatin, clotrimazole, fluconazole, ciclopirox, econazole, naftifine, terbinafine and griseofulvin.

19. The peptide-cargo complex of claim 7, wherein the cargo is an antineoplastic agent selected from the group consisting of pentostatin, pentamidine, 6-mercaptopurine, 6-thioguanine, methotrexate, bleomycins, etoposide, teniposide, dactinomycin, daunorubicin, doxorubicin, mitoxantrone, hydroxyurea, 5-fluorouracil, cytarabine, fludarabine, mitomycin, cisplatin, procarbazine, dacarbazine, paclitaxel, colchicine, and vinca alkaloids.

20. The peptide-cargo complex of claim 7, wherein the cargo is an immunosuppressive agent selected from the group consisting of methotrexate, azathioprine, fluorouracil, hydroxyurea, 6-thioguanine, cyclophosphamide, mechloroethamine hydrochloride, carmustine, cyclosporine, taxol, tacrolimus, vinblastine, dapsone, nedocromil, cromolyn (cromoglycic acid), and sulfasalazine.

21. The peptide-cargo complex of claim 7, wherein the cargo is an analgesic agent selected from the group consisting of lidocaine, bupivacaine, novocaine, procaine, tetracaine, benzocaine, cocaine, mepivacaine, etidocaine, proparacaine, ropivacaine, and prilocaine.

22. The peptide-cargo complex of claim 7, wherein the cargo is a hormone selected from the group consisting of tissue hormones, gastrointestinal hormones, releasing hormones, pituitary hormones, insulin, vasopressin (ADH), glucagon, enkephalin, calcitonin, and corticosteroides.

23. The peptide-cargo complex of claim 7, wherein the cargo is a histamine receptor agonist or antagonist selected from 2-methylhistamine, 2-pyridylethylamine, 2-thiazolyl-ethylamine, (R)-a-methylhistamine, impromidine, dimaprit, 4(5)methyl-histamine, diphenhydramine, pyrilamine, promethazine, chlorpheniramine, chlorcyclizine, and terfenadine.

24. The peptide-cargo complex of claim 7, wherein the cargo is an cytokine or growth factor selected from the group consisting of IL-1Ra, STNF-R (p55), STNF-R (p75), SIL-1R type I, SIL-1R type II, BMP-2, BMP-6, BMP-7, LMP-1, LMP-3, IGF-1, TGF-β1, TGF-β2, TGF-β3, IL-4, IL-10, CTLA4, CD30, TIMP-1, IFN-β, Sox-9, and PDGF.

25. The peptide-cargo complex of claim 7, wherein the cargo is an antiaging agent selected from the group consisting of retinoic acid, hyaluronic acid, collagen, and free radical catchers.

26. The peptide-cargo complex of claim 7, wherein the cargo is a therapeutic agent for a condition selected from the group consisting of Crohn's disease, ulcerative colitis, gastrointestinal ulcers, peptic ulcer disease, and abnormal proliferative diseases.

27. The peptide-cargo complex of claim 7, wherein the cargo is a therapeutic for ulcers and is selected from the group consisting of an H2-histamine inhibitor, an inhibitor of the proton-potassium ATPase, and an antibiotic directed at Helicobacter pylori.

28. The peptide-cargo complex of claim 7, wherein the cargo is a therapeutic agent for treating a bronchial condition selected from cystic fibrosis, asthma, allergic rhinitis, and chronic obstructive pulmonary disease.

29. The peptide-cargo complex of claim 7, wherein the cargo is a therapeutic agent for treating a condition selected from the group consisting of ischemia, Parkinson's disease, schizophrenia, cancer, acquired immune deficiency syndrome (AIDS), infections of the central nervous system, epilepsy, multiple sclerosis, neurodegenerative disease, trauma, depression, Alzheimer's disease, migraine, pain, and a seizure disorder.

30. The peptide-cargo complex of claim 7, wherein the cargo is a therapeutic agent for a condition selected from the group consisting of inflammatory and degenerative joint and spine diseases, arthritis, especially osteoarthritis, low back pain, bone repair, fracture healing, therapy of muscle and ligament injury.

31. A method for cellular internalisation of a cargo molecule which comprises linking a cargo molecule to a peptide according to claim 1 to form a cargo molecule-peptide complex.

32. A method for producing a kit for cellular internalisation of a cargo molecule in an animal or a human body which method comprises linking a cargo molecule to a peptide according to claim 1 to form a cargo molecule-peptide complex and producing a kit comprising said cargo molecule-peptide complex.

33. A method for nuclear translocation of a cargo molecule in a target cell which comprises linking a cargo molecule to a peptide according to claim 1.

34. A method for producing a kit for nuclear translocation of a cargo molecule in a target cell in an animal or a human body which comprises linking a cargo molecule to a peptide according to claim 1 to form a cargo molecule-peptide complex and producing a kit comprising said cargo molecule-peptide complex.

35. A method for translocation of a cargo molecule in the mitochondria of a target cell which comprises linking said cargo molecule to a peptide according to claim 1.

36. A method for producing a kit for translocation of a cargo molecule in the mitochondria of a target cell in an animal or a human body which comprises linking said cargo molecule to a peptide according to claim 1 to form a cargo molecule-peptide complex and producing a kit comprising said cargo molecule-peptide complex.

37. A method for the treatment of a condition selected from the group consisting of Crohn's disease, ulcerative colitis, gastrointestinal ulcers, peptic ulcer disease, abnormal proliferative diseases, an infection with Helicobacter pylori, cystic fibrosis, asthma, allergic rhinitis, chronic obstructive pulmonary disease, ischemia, Parkinson's disease, schizophrenia, cancer, acquired immune deficiency syndrome (AIDS), infections of the central nervous system, epilepsy, multiple sclerosis, neurodegenerative disease, trauma, depression, Alzheimer's disease, migraine, pain, and a seizure disorder which method comprises administering to an individual in need thereof having at least one said condition, an effective amount of cargo molecule for treating said condition, which is linked to a peptide according to claim 1.

38. (canceled)

39. A method for treating a condition selected from the group consisting of glucocerebrosidase deficiency (Gaucher's disease), mucopolysaccharidosis I, sanfilippo B syndrome, pancreatic insufficiency, severe combined immunodeficiency syndrome, and neuromuscular dysfunction associated with triose phosphate isomerase deficiency which method comprises administering to an individual in need thereof having at least one said condition, an effective amount of a cargo molecule for treating said condition, which is linked to a peptide according to claim 1.

40. (canceled)

41. A method for treating a condition selected from the group consisting of inflammatory and degenerative joint and spine diseases, arthritis, especially osteoarthritis, low back pain, bone repair, fracture healing, therapy of muscle and ligament injury which method comprises administering to an individual in need thereof having at least one said condition, an effective amount of cargo molecule for treating said condition which is linked to a peptide according to claim 1.

42. (canceled)

43. A pharmaceutical composition comprising:
a cargo-peptide complex according to claim 7, comprising an effective amount of a biologically active or therapeutic agent, and
a pharmaceutically acceptable carrier.

44. A method for the delivery of a cargo to the surface of, into or across a biological barrier, comprising:
a) providing a cargo and at least one delivery peptide according to claim 1,
b) forming a peptide-cargo complex,
c) contacting the barrier with the peptide-cargo complex, and
d) delivering the cargo to the surface of, into or across the barrier.

45. The method of claim 44, wherein the barrier is an intact epithelial or endothelial tissue layer or layers.

46. The method of claim 44, wherein the barrier is skin.

47. The method of claim 46, wherein the cargo is delivered into and/or across at least one layer selected from the group consisting of stratum corneum, stratum granulosum, stratum lucidum, and stratum germinativum.

48. The method of claim 46, wherein the contacting of the skin with the peptide-cargo complex is accomplished by administering a composition comprising the peptide-cargo complex topically to the skin.

49. The method of claim 48, wherein the composition is in a form selected from the group consisting of a cream, ointment, salve, lotion, and a transdermal patch.

50. The method of claim 44, wherein the barrier is the gastrointestinal tract.

51. The method of claim 44, wherein the barrier is the pulmonary epithelium.

52. The method of claim 44, wherein the barrier is the endothelial blood brain barrier.

53. A method for inducing synovial cell death comprising administering the peptide-cargo complex of claim 7 to said synovial cell.

54. (canceled)

55. A method for inducing apoptosis in a tumor cell comprising administering the peptide-cargo complex of claim 7 to said tumor cell.

56. (canceled)

57. A method for reducing white blood cells in arthritic joints comprising administering the peptide-cargo complex of claim 7 to said white blood cells.

58. (canceled)

59. A method for reducing the effects of skin aging comprising administering to one in need thereof the peptide-cargo complex of claim 7 to the skin.

60. (canceled)

61. (canceled)

62. A method for eliciting an immune response in an animal or a human body comprising administering to a target cell of said body an immunogen comprising the peptide-cargo complex of claim 7.

63. A method for producing an immunogen for eliciting an immune response in an animal or a human body which method comprises forming a peptide-cargo complex according to claim 7.
